(19) United States
(12) Patent Application Publication
(10) Pub. No.: US 2016/0095995 A1
(43) Pub. Date: Apr. 7, 2016

(54) BITE BLOCK

(71) Applicant: Haider Biologics LLC, San Diego, CA (US)

(72) Inventors: Thomas T. Haider, Rancho Santa Fe, CA (US); Gustavo R. Prado, San Diego, CA (US)

(21) Appl. No.: 14/933,320

(22) Filed: Nov. 5, 2015

Related U.S. Application Data
(63) Continuation-in-part of application No. 14/275,672, filed on May 12, 2014, now Pat. No. 9,233,221.
(60) Provisional application No. 61/822,801, filed on May 13, 2013.
(60) Provisional application No. 61/837,074, filed on Jun. 19, 2013.
(60) Provisional application No. 61/846,993, filed on Jul. 16, 2013.
(60) Provisional application No. 62/078,892, filed on Nov. 12, 2014.

(51) Int. Cl. A61M 16/04

(52) U.S. Cl. CPC A61M 16/0493

(57) ABSTRACT

A bite block and methods of use thereof are disclosed. The bite block includes a body and a tube clamp attached thereto. The bite block may be configured to encompass a tube such as an endotracheal tube and another medical device such as a body temperature probe, and to protect the mouth of an intubated patient from clenching damage.

### CROSS-REFERENCE

This application is a continuation-in-part of U.S. Non-provisional application Ser. No. 14/275,672, filed on May 12, 2014, which claim the benefit of U.S. Provisional Application No. 61/822,801, filed May 13, 2013, U.S. Provisional Application No. 61/837,074, filed Jun. 19, 2013, and U.S. Provisional Application No. 61/846,993, filed Jul. 16, 2013; and also claims priority to U.S. Provisional Application No. 62/078,892 filed Nov. 12, 2014, each of which is hereby explicitly incorporated herein by reference in its entirety.

### BACKGROUND

Many current bite blocks and mouth guards used in intubated patients do not provide anchoring for the endotracheal tube while protecting the teeth and soft tissues of the mouth from clenching damage against one another or against the bite block or mouth guard. Injuries to the teeth or soft tissues of the mouth are common in procedures where intubation occurs, especially if the procedure requires patient repositioning such as in orthopedic surgeries. In addition, endotracheal tube migration can be life-threatening if it goes unnoticed during surgery.

### SUMMARY OF THE INVENTION

Provided herein are embodiments related to a bite block to be used by a health care provider and more particularly to a bite block that can be secured to an endotracheal tube or similar device deployed at multiple positions in the mouth of a patient.

The current disclosure relates to devices and methods comprising a clamp and a body, such as Surgical Bite Block, and use thereof, for example to hold in place a device such as an endotracheal tube. The tube clamp is generally made from a flexible material such as nylon or acetal plastic with or without fiberglass reinforcement or other reinforcement such as medical grade plastic. The tube clamp generally clamps around an endotracheal tube such that the invention is affixed to the endotracheal tube. The tube clamp is generally positioned outside the mouth of the patient when properly deployed, although embodiments wherein the tube clamp is positioned within the mouth of a patient when the device is deployed are also contemplated. The body is generally made from a soft material, such as silicone or a medical grade foam. In some embodiments, when properly deployed, the body preferentially rests inside the mouth of the patient thereby anchoring the invention inside the patient's mouth. The bite block anchors the endotracheal tube inside the patient, prevents it from migrating and protects the mouth and teeth of the patient.

Some embodiments relate to a bite block comprising: a body comprising an outer surface and a first portion sized and shaped to fit in the mouth of a person; an endotracheal tube clamp sized to fit accommodate an endotracheal tube therein and coupled to the body; a first channel running through the body sized to fit accommodate the endotracheal tube therein; and a slit extending from the outer surface of the body and joining the first channel that allows the endotracheal tube to be placed in the first channel or removed from the first channel. In some aspects the first channel is round or ovoid. In some aspects, the body comprises an outer surface. In some aspects, the bite block further comprises a slit extending from the outer surface of the body and joining the first channel. In some aspects, the slit allows an endotracheal tube to be placed in the first channel. In some aspects, the slit allows an endotracheal tube to be removed from the first channel. In some aspects, the slit is bounded by rounded edges on the outer surface of the body. In some aspects, the rounded edges protect the lips of a patient from pinching damage when the body is applied to the patient's mouth. In some aspects, the body further comprises a second channel running through the body. In some aspects, the second channel joins the slit. In some aspects, the second channel is round or ovoid. In some aspects, a first arm that connects the body is coupled to the endotracheal tube clamp by a first arm. In some aspects, the first arm is at least one of: molded into the body, attached to the body with screws, and attached to the body with adhesive. In some aspects, the first arm is made out of a flexible material. In some aspects, the flexible material is a medical grade plastic. In some aspects, the medical grade plastic is at least one of acetal, and nylon plastic. In some aspects, the medical grade plastic is nylon plastic. In some aspects, the medical grade plastic is supplemented with 20%-40% fiberglass. In some aspects, the medical grade plastic is supplemented with 30% fiberglass. In some aspects, the body is coupled to the endotracheal tube clamp by a second arm. In some aspects, the second arm is at least one of: molded into the body, attached to the body with screws, and attached to the body with adhesive. In some aspects, the bite block comprises a neck strap connection. In some aspects, the neck strap connection is attached to an arm. In some aspects, the neck strap connection is molded into an arm. In some aspects, the neck strap connection is attached to the endotracheal tube clamp. In some aspects, the neck strap connection is attached to the body. In some aspects, the neck strap connection is at least one of a ring, a hollow rectangle, a slit or a post. In some aspects, the bite block further comprises a finger support. In some aspects, the finger support is a part of the first arm. In some aspects, a neck strap connection is a part of the finger support. In some aspects, the finger support is attached to the first arm. In some aspects, the finger support is either a ring or a hollow rectangle. In some aspects, the body is at least one of spherical, cylindrical, square, rectangular, or ovoid shape. In some aspects, the body is made of a soft material. In some aspects, the soft material is Silicone. In some aspects, the soft material is a medical grade foam. In some aspects, the endotracheal tube clamp comprises a locking mechanism. In some aspects, the locking mechanism has a single locking position. In some aspects, the locking mechanism has multiple locking positions. In some aspects, the locking mechanism comprises a latch. In some aspects, the latch comprises at least one of and a hook and a notch, a hook and a plurality of notches, or a pair of hooks. In some aspects, the locking mechanism comprises at least one of a pair of magnets, a zip tie, a reversible zip tie, and a nut and bolt. In some aspects, the locking mechanism comprises a pair of handles for opening and closing the locking mechanism. In some aspects, the endotracheal tube clamp comprises a series of ridges on an interior of the clamp. In some aspects, the endotracheal tube is made of a flexible material. In some aspects, the flexible material is a medical grade plastic. In some aspects, the medical grade plastic is acetal plastic. In some aspects, the body comprises a first groove for a patient's teeth or gums. In some aspects, the body comprises a second groove for the patient's teeth or gums opposite the first groove. In some aspects, the first groove extends around the circumference of the body. In some aspects, the first groove and the second groove extend into the first channel. In some aspects, the body comprises an anterior aspect and a posterior aspect. In some aspects, the posterior aspect is at least one of bulbous, cylindrical, and spherical. In some aspects, the anterior aspect is at least one of cylindrical or spherical.

Some embodiments relate to a bite block comprising: a bipartite body comprising a first body piece and a second body piece separated by a slit and sized and shaped to fit in combination into a mouth of a patient, an endotracheal tube clamp, and a first channel running through the body. In some aspects, the first body piece and the second body piece are held together by the endotracheal tube clamp. In some aspects, the endotracheal tube clamp has a first locking mechanism and a second locking mechanism. In some aspects, at least one of the first locking mechanism and the second locking mechanism comprises a latch. In some aspects, the latch comprises at least one of and hook and a notch, a hook and a plurality of notches, and a pair of hooks. In some aspects, at least one of the first locking mechanism and the second locking mechanism comprises at least one of a pair of magnets, a zip tie, a reversible zip tie, and a nut and bolt. In some aspects, the first channel is either round or ovoid. In some aspects, the body further comprises a second channel. In some aspects, the second channel intersects the first channel. In some aspects, the second channel is either round or ovoid. In some aspects, the body is made of a soft material. In some aspects, the soft material is silicone. In some aspects, the soft material is a medical grade foam. In some aspects, at least one of the first locking mechanism the second locking mechanism comprises a pair of handles for opening and closing the locking mechanism. In some aspects, the endotracheal tube clamp comprises a series of ridges on an interior of the clamp. In some aspects, the endotracheal tube is made of a flexible material. In some aspects, the flexible material is a medical grade plastic. In some aspects, the medical grade plastic is acetal plastic. In some aspects, the body comprises a first groove for a patient's teeth or gums. In some aspects, the body comprises a second groove for the patient's teeth or gums opposite the first groove. In some aspects, the first groove extends around the circumference of the body. In some aspects, the first groove and the second groove extend into the first channel. In some aspects, the body comprises an anterior aspect and a posterior aspect. In some aspects, the posterior aspect is at least one of bulbous, cylindrical, and spherical. In some aspects, the anterior aspect is at least one of cylindrical spherical.

Some embodiments relate to methods of protecting a patient's mouth from clenching damage during endotracheal tube administration, comprising the steps of surrounding a segment of an endotracheal tube with a soft body held to the endotracheal tube by at least a clamp, and deploying the body to the interior of a patient's mouth. In some aspects, said clamp may be reversibly released from said endotracheal tube. In some aspects, said clamp may be configured to engage said endotracheal tube in a plurality of clamp positions. In some aspects, said endotracheal tube may be adjusted when released from said clamp. In some aspects, said endotracheal tube may be deployed to a patient prior to holding said soft body to the endotracheal tube. In some aspects, said clamp grasps said endotracheal tube via a plurality of grooves in an interior of said clamp. In some aspects, said body is held in place by a neck strap holding said body in place in said patient's mouth. In some aspects, said body comprises at least one groove to accommodate patient teeth or gums, and wherein said body is held in place by a fit of said patient teeth or gums into said groove or grooves.

I Some embodiments relate to a bite block comprising a body comprising a posterior aspect, a first groove, a second groove, and an anterior portion, wherein said body comprises silicone or medical grade foam; a first channel running through the body from an external side of the body to a mouth side of the body through the posterior aspect, the first channel is sized to accommodate an endotracheal tube therein; a slit extending from the first channel to an outer surface of the body through the posterior aspect, such that the bite block can be deployed onto an endotracheal tube after the endotracheal tube is intubated into a person; and wherein said posterior aspect is deployed proximal to a subset of teeth or gums of the person, wherein the subset of teeth or gums of the person rest in the grooves, wherein the posterior aspect has a length at least as long as the length of the groove and has a height at least two times the height of the groove, wherein the posterior aspect comprises a shape having each dimension greater than the cross-sectional diameter of the first channel. In some aspects, the bite block comprises an endotracheal tube clamp coupled to the body, sized to accommodate an endotracheal tube therein. In some aspects the bite block occupies less than two-thirds of an adult mouth opening. In some aspects, the bite block occupies less than one-third of an adult mouth opening. In some aspects, the posterior aspect has a width less than the distance measured between two upper or two lower bicuspids in an adult mouth. In some aspects, the bite block is operably deployable in a first position and a second position within the mouth of the person. In some aspects, the bite block, when operably deployed into the mouth of the person, does not block access to the oral cavity of the person by a health care professional. In some aspects, the bite block, when operably deployed into the mouth of the person, does not block a line of sight to the oral cavity of the person. In some aspects, the first groove and second groove are configured to fit any adjacent subset of teeth of the person. In some aspects, the first groove and the second groove are neutral with respect to the teeth that rest in the groove. In some aspects, the posterior aspect and the grooves distribute clenching force over an area of teeth, gums, teeth and gums, or palate of the person that is greater than an area of clenching contact between the person's upper and lower jaws. In some aspects, the posterior aspect distributes clenching force beyond an initial point of contact between the person's upper and lower teeth, gums, or teeth and gums. In some aspects, the posterior aspect distributes clenching force to the person's palate when properly deployed. In some aspects, the posterior aspect comprises a material having a durometer measurement of from 10 to 80. In some aspects, the posterior aspect comprises at least one material selected from the list consisting of urethane foam, polyurethane foam, ethylene foam, polyethylene foam, thermoplastic elastomer and silicone. In some aspects, the posterior aspect is reversibly deformable under clenching force. In some aspects, the posterior aspect comprises a shape that is at least one of bulbous, cylindrical, and spherical. In some aspects, the bite block comprises a second channel.

In some embodiments, provided herein is a device for concurrently protecting the teeth and gums of a person and providing access to the mouth of the person, wherein the device comprises a silicone or medical grade foam body comprising a posterior aspect, a first groove, a second groove, and an anterior portion; a first channel running through the body from an external side of the body to a mouth side of the body through the posterior aspect, the first channel sized to accommodate an endotracheal tube therein; a slit extending from the first channel to an outer surface of the body through the posterior aspect, such that the bite block can be deployed onto an endotracheal tube after the endotracheal tube is intubated into the person; and wherein said posterior aspect has a length at least as long as the length of the groove and has a height at least two times the height of the groove, and wherein the posterior aspect comprises a shape having each dimension greater than the cross-sectional diameter of the first channel. In some aspects, the device further comprises an endotracheal tube clamp coupled to the body or integral with the body, sized to accommodate an endotracheal tube therein. In some aspects, the bite block occupies less than two-thirds of an adult mouth opening. In some aspects, the bite block occupies less than one-third of an adult mouth opening. In some aspects, the posterior aspect has a width less than the distance measured between two upper or two lower bicuspids in an adult mouth. In some aspects, the device is operably deployable in a first position and a second position within the mouth of the person. In some aspects, the device, when operably deployed into the mouth of the person, does not block access to the oral cavity of the person by a health care professional. In some aspects, the device, when operably deployed into the mouth of the person, does not block a line of sight to the oral cavity of the person. In some aspects, the first groove and second groove are configured to fit any adjacent subset of teeth of the person. In some aspects, the first groove and the second groove are neutral with respect to the teeth that rest in the groove. In some aspects, the posterior aspect and the grooves distribute clenching force over an area of teeth, gums, teeth and gums, or palate of the person that is greater than an area of clenching contact between the person's upper and lower jaws. In some aspects, the posterior aspect distributes clenching force beyond an initial point of contact between the person's upper and lower teeth, gums, or teeth and gums. In some aspects, the posterior aspect distributes clenching force to the person's palate when properly deployed. In some aspects, the posterior aspect comprises a material having a durometer measurement of from 10 to 80. In some aspects, the posterior aspect comprises at least one material selected from the list consisting of urethane foam, polyurethane foam, ethylene foam, polyethylene foam, thermoplastic elastomer and silicone. In some aspects, the posterior aspect is reversibly deformable under clenching force. In some aspects, the posterior aspect comprises a shape that is at least one of bulbous, cylindrical, and spherical. In some aspects, the device comprises a second channel.

In some embodiments, provided herein is a method for protecting the teeth and gums from clenching damage in a person requiring endotracheal tube administration, comprising the steps of surrounding a segment of an endotracheal tube with a bite block held to the endotracheal tube and deploying a posterior aspect of the bite block to the interior of the person's mouth, wherein the posterior aspect distributes clenching force over an area of teeth, gums, teeth and gums, or palate of the person that is greater than an area of clenching contact between the person's upper and lower jaws during intubation in absence of the bite block. In some aspects, the bite block contacts the palate of the person before the bite block contacts the teeth of the person in an unclenching mouth. In some aspects, the posterior aspect is compressed before the person's teeth contact the bite block. In some aspects, the clenching force is distributed over a first area at a first force of clenching and the clenching force is distributed over a second area at a second force of clenching, wherein the second force of clenching is greater than the first force of clenching, and wherein the pressure per unit area is not linear with increasing force.

In some embodiments, provided herein is a bite block configured to increase surface area of contact with a mouth of a person as clenching force increases. In some aspects, the bite block comprises a body comprising a posterior aspect, a first groove, a second groove, and an anterior portion, wherein said body comprises silicone or medical grade foam. In some aspects, the bite block comprises a first channel running through the body from an external side of the body to a mouth side of the body through the posterior aspect, the first channel is sized to accommodate an endotracheal tube therein. In some aspects, the bite block comprises a slit extending from the first channel to an outer surface of the body through the posterior aspect, such that the bite block can be deployed onto an endotracheal tube after the endotracheal tube is intubated into the person. In some aspects, the posterior aspect is deployed proximal to a subset of teeth or gums of the person, wherein the subset of teeth or gums of the person rest in the grooves, wherein the posterior aspect has a length at least as long as the length of the groove and has a height at least two times the height of the groove, wherein the posterior aspect comprises a shape having each dimension greater than the cross-sectional diameter of the first channel. In some aspects, the bite block comprises an endotracheal tube clamp coupled to the body, sized to accommodate an endotracheal tube therein. In some aspects, the bite block occupies less than two-thirds of an adult mouth opening. In some aspects, the bite block occupies less than one-third of an adult mouth opening. In some aspects, the bite block has a width less than the distance measured between two upper or two lower bicuspids in an adult mouth. In some aspects, the bite block is operably deployable in a first position and a second position within the mouth of the person. In some aspects, the bite block, when operably deployed into the mouth of the person, does not block access to the oral cavity of the person by a health care professional. In some aspects, the bite block, when operably deployed into the mouth of the person, does not block a line of sight to the oral cavity of the person. In some aspects, the first groove and second groove are configured to fit any adjacent subset of teeth of the person. In some aspect, the first groove and the second groove are neutral with respect to the teeth that rest in the groove. In some aspects, the posterior aspect and the grooves distribute clenching force over an area of teeth, gums, teeth and gums, or palate of the person that is greater than an area of clenching contact between the person's upper and lower jaws. In some aspects, the posterior aspect distributes clenching force beyond an initial point of contact between the person's upper and lower teeth, gums, or teeth and gums. In some aspects, the posterior aspect distributes clenching force to the person's palate when properly deployed. In some aspects, the posterior aspect comprises a material having a durometer measurement of from 10 to 80. In some aspects, the posterior aspect comprises at least one material selected from the list consisting of urethane foam, polyurethane foam, ethylene foam, polyethylene foam, thermoplastic elastomer and silicone. In some aspects, the posterior aspect is reversibly deformable under clenching force. In some aspects, the posterior aspect comprises a shape that is at least one of bulbous, cylindrical, and spherical. In some aspects, the bite block comprises a second channel.

In some embodiments, provided herein is a device for the mouth of an intubated person comprising teeth-independent grooves, posterior body, channel through the posterior body, configured to accommodate an endotracheal tube. In some aspects, the posterior body has a length at least as long as the length of the grooves and has a height at least two times the height of the grooves, wherein the posterior body comprises a shape having each dimension greater than the cross-sectional diameter of the first channel and the channel is sized to accommodate an endotracheal tube therein. In some aspects, the device comprises an endotracheal tube clamp coupled to an anterior body, sized to accommodate an endotracheal tube therein. In some aspects, the device comprises a slit extending from the first channel to an outer surface of the device through the posterior body, such that the device can be deployed onto an endotracheal tube after the endotracheal tube is intubated into the person. In some aspects, the device occupies less than two-thirds of an adult mouth opening. In some aspects, the device occupies less than one-third of an adult mouth opening. In some aspects, the posterior body has a width less than the distance measured between two upper or two lower bicuspids in an adult mouth. In some aspects, the device is operably deployable in a first position and a second position within the mouth of the person. In some aspects, the device, when operably deployed into the mouth of the person, does not block access to the oral cavity of the person by a health care professional. In some aspects, the device, when operably deployed into the mouth of the person, does not block a line of sight to the oral cavity of the person. In some aspects, the first and second grooves are configured to fit any adjacent subset of teeth of the person. In some aspects, the posterior body and the grooves distribute clenching force over an area of teeth, gums, teeth and gums, or palate of the person that is greater than an area of clenching contact between the person's upper and lower jaws. In some aspects, the posterior body distributes clenching force beyond an initial point of contact between the person's upper and lower teeth, gums, or teeth and gums. In some aspects, the posterior body distributes clenching force to the person's palate when properly deployed. In some aspects, the posterior body comprises a material having a durometer measurement of from 10 to 80. In some aspects, the posterior body and grooves comprise at least one material selected from the list consisting of urethane foam, polyurethane foam, ethylene foam, polyethylene foam, thermoplastic elastomer and silicone. In some aspects, the posterior body is reversibly deformable under clenching force. In some aspects, the posterior body comprises a shape that is at least one of bulbous, cylindrical, and spherical. In some aspects, the device comprises a second channel.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an embodiment of the surgical bite block.

FIG. 2A shows an embodiment of the endotracheal tube clamp in the unlocked position.

FIG. 2B shows an embodiment of the endotracheal tube clamp in the locked position.

FIG. 2C presents an alternate view of the endotracheal tube clamp in the unlocked position as in FIG. 2A.

FIG. 3 shows the placement of an embodiment of the surgical bite block around an endotracheal tube.

FIG. 4A shows an embodiment of the body without grooves for the patient's teeth and gums.

FIG. 4B shows an embodiment of the body with grooves for the patient's teeth and gums.

FIG. 4C shows an embodiment of the body with grooves for the patient's teeth and gums.

FIG. 5A shows an embodiment of the body with a bulbous shaped posterior aspect without grooves for the patient's teeth and gums.

FIG. 5B shows an embodiment of the body with a bulbous shaped posterior aspect with grooves.

FIG. 6 shows a further embodiment of the body with a bulbous shaped posterior aspect.

FIG. 7 shows an embodiment of a clamp lacking a body and having struts to which a body is affixed.

FIG. 8 shows an embodiment of an endotracheal tube clamp having four hooks and notches to accommodate a greater range endotracheal tube sizes.

FIG. 9A shows dimensions of various elements of a bite block.

FIG. 9B shows dimensions of various elements of a bite block.

### DETAILED DESCRIPTION OF THE INVENTION

The disclosure herein relates to devices and methods related to the protection of a patient's mouth and face during intubation procedures. Some embodiments relate to bite blocks. In some cases, a bite block comprises: a body comprising an outer surface and a first portion sized and shaped to fit in the mouth of a person; optionally an endotracheal tube clamp sized to accommodate an endotracheal tube therein and coupled to the body; a first channel running through the body sized to fit accommodate the endotracheal tube therein; and a slit extending from the outer surface of the body and joining the first channel that allows the endotracheal tube to be placed in the first channel or removed from the first channel.

Disclosed herein are bite blocks comprising: a body sized and shaped to fit in a mouth of a person, wherein said body comprises a soft material such as silicone, medical grade foam, or thermoplastic, and wherein said body comprises: a) a first groove and a second groove into which teeth of said patient may fit to stabilize said body when said body is deployed into the mouth of said patient, b) a first channel running through the body sized to accommodate the endotracheal tube therein, c) a second channel sized to accommodate a measuring device, running parallel to said first channel within said body, and d) a slit extending from an outer surface of the body and joining the first channel said slit comprising rounded edges at said outer surface to protect the mouth of said person when said body is deployed into the mouth of said patient; and wherein said bite block further comprises an endotracheal tube clamp sized to accommodate an endotracheal tube therein and coupled to the body, e) wherein said endotracheal tube comprises a locking mechanism, said locking mechanism comprising a latch, said latch comprising a hook and at least one notch in an opposing configuration relative to said hook such that said hook and said notch may engage to hold said latch in a closed configuration, f) wherein said latch in said closed configuration is capable of holding an endotracheal tube passed there through substantially in place, and g) wherein said latch in a second open configuration does not comprise said hook engaged with said notch, and h) wherein said latch in said open configuration is not capable of holding an endotracheal tube clamp passed there through substantially in place, i) said endotracheal tube clamp further comprising openings through which a neck strap may be threaded to hold said endotracheal tube clamp and said body in place when said body is deployed into the mouth of a person.

Also disclosed herein are methods of protecting a patient's mouth from clenching damage during endotracheal tube administration, comprising the steps of surrounding a segment of an endotracheal tube with a soft body held to the endotracheal tube by at least a clamp, and deploying the body to the interior of a patient's mouth. Also disclosed herein are methods for protecting the teeth and gums from clenching damage in a person requiring endotracheal tube administration, comprising the steps of surrounding a segment of an endotracheal tube with a bite block held to the endotracheal tube and deploying a posterior aspect of the bite block to the interior of the person's mouth, wherein the posterior aspect distributes clenching force over an area of teeth, gums, teeth and gums, or palate of the person that is greater than an area of clenching contact between the person's upper and lower jaws during intubation in absence of the bite block.

Some embodiments of the bite block comprise one or more of the following elements.

### Body

Bite blocks disclosed herein comprise a body capable of being inserted into the mouth of a patient and deployed at multiple positions within a patient's mouth. A number of body shapes are consistent with the disclosure herein. In some embodiments the body is round, cylindrical, oval, lozenge-shaped, bulbous, a cylindrical ellipse, a cylindrical hexagon, a cylindrical octagon, a spherical segment, a cylindrical segment, rectangular, square, barrel-shaped, or other shape consistent with insertion into a patient's mouth. Some bite block bodies are spherical. Some bite block bodies are cylindrical. Some bite block bodies are ovoid. Some bite block bodies are bulbous. Some bite block bodies have any shape consistent with this element is contemplated. Alternative body shapes consistent with the disclosure herein share the element of capability of being inserted into the mouth cavity of a patient.

In some embodiments the body is bilaterally symmetrical along its long axis, or otherwise along an axis parallel with the long axis of a patient's throat cavity.

In some embodiments the body comprises a first channel running through its center. In some embodiments the first channel is sized and shaped to accommodate a tube, such as an endotracheal tube, to pass there through. In some embodiments the diameter of the first channel ranges from 2 to 15 mm, for example 2, 3, 4, 5, 6, 7, 8, 9, 10, 11, 12, 13, 14, or 15 mm, or any non-integer value within said range of 2 to 15 mm. In some embodiments the channel is accompanied by a second channel, such as a second channel in parallel or intersecting the first channel, for example configured to accommodate a device such as a temperature probe, light, camera, or other device required to be inserted through the mouth or oral cavity during surgery. In some embodiments, the first channel is sized, shaped or sized and shaped to allow at least one other device such as at least one temperature probe to pass through the first channel. The first channel is circular in cross section in some cases. The first channel is elliptical in cross section in some cases. In some embodiments the first channel is triangular, rectangular, pentagonal, hexagonal, heptagonal, octagonal, or demonstrates alternative regular or irregular geometry in cross section. In some embodiments only an endotracheal tube is able to pass through the first channel.

In some embodiments the body is divided along or parallel to its long axis or otherwise along the channel discussed above. In some embodiments the body is divided so as to allow the body to be split apart and reconstituted around a tube, such as an endotracheal tube deployed to a patient. In some embodiments the tube such as an endotracheal tube is positioned to pass through a first channel of the body. In some embodiments the body is divided from one surface through to a first central channel, while in other embodiments the body is divided from one surface through to a second surface, so that it is partially or totally split into halves or unequal fractions. In some embodiments, the edges of the body are rounded or otherwise softened so that reconstitution of the body within the mouth of a patient does not pinch or harm patient mouth tissue.

In some embodiments a second channel is connected to the first channel. In some embodiments, the second channel intersects the first channel. In some embodiments the second channel allows at least one other device such as at least one temperature probe to pass through the body. In some embodiments, the bite block comprises a slit on the body. In some embodiments the slit is coplanar with the first channel. In some embodiments the slit is on the superior side of the body. In some embodiments, the slit is on the inferior side of the body. In some embodiments, the slit extends into the first channel. In some embodiments the slit allows an endotracheal tube or other devices to slide through the slit and into the first channel. In some embodiments, the slit, intersects the second channel. In some embodiments, the slit extends completely through the first channel and divides the body into two pieces. In some embodiments the two pieces of the body are held together by the endotracheal tube clamp.

The body is made of a soft, durable substance. Some bite block bodies are made of silicone. Some bite block bodies are made of a synthetic foam such as a medical grade foam. In some embodiments the body is made of a urethane foam. In some embodiments, the body is made of a polyurethane foam. In some embodiments, the body is made of an ethylene foam. In some embodiments, the body is made of a polyethylene foam. In some embodiments, the body is made of a thermoplastic elastomer. In some embodiments, the body is made of a material with characteristics similar thereto any of the above materials.

In embodiments having an endotracheal tube clamp, the body connects to the endotracheal tube clamp at a first arm or a first arm and a second arm. In some embodiments the body is molded around the first arm or the first and second arms. In some embodiments, the first arm or the first and second arms are attached to the body with one or more screws. In some embodiments, the first arm or the first and second arms are attached to the body with an adhesive. In some embodiments, the first arm or the first and second arms are made of a flexible material. In some embodiments the first arm or the first and second arms are made of a flexible material such as acetal, nylon, polypropylene, polycarbonate, or acrylic plastic, or other medical grade plastic. In some embodiments the acetal, nylon, polypropylene, polycarbonate, or acrylic plastic, or other medical grade plastic is reinforced with fiberglass reinforcement or other medical grade plastic. In some embodiments the reinforcement is present at 20, 21, 22, 23, 24, 25, 26, 27, 28, 29, 30, 31, 32, 33, 34, 35, 36, 37, 38, 39, or 40%, or any non-integer value within a range of 20% to 40%. In some embodiments the flexible material is nylon, reinforced with 30% fiberglass. In some embodiments, the first arm or the first and second arms and are shaped and positioned such that they allow concurrent opening of the endotracheal tube clamp and the body, for example to ease the placement of the endotracheal tube. In some embodiments the first arm or the first and second arms are curved. In some embodiments, the first arm or the first and second arms are S shaped.

In some embodiments, the device comprises a neck strap having ends and a neck strap connection. In some embodiments, the neck strap connection is attached to the first arm 30a or the first and second arms. In some embodiments, the neck strap connection is built into the first arm. A number of neck strap configurations are consistent with the disclosure herein. In some embodiments, the neck strap connections are each a ring. In some embodiments the neck strap connections are each rectangular. In some embodiments, the neck strap connections are each D-shaped. In some embodiments, the neck strap connections are each a clip. In some embodiments, the neck strap connections are each a post. In some embodiments, the neck strap having ends fits around the patient's neck to further secure the device to the patient.

In some embodiments the body comprises a groove on its top surface relative to a standing patient, its bottom surface, or its top and its bottom surfaces. In some embodiments the top groove accommodates a patient's teeth or gums when the body is deployed into a patient mouth. In some embodiments the bottom groove accommodates a patient's bottom teeth when the body is deployed into a patient mouth. In some embodiments, the top and or bottom groove aligns with a patient's teeth or gums. In some embodiments the bite block comprises a body with a first groove. In some embodiments, the patient's teeth or gums rest within the first groove, further securing the body in the patient's mouth when the body is properly deployed into a patient's mouth. In some embodiments, the body has a second groove. In some embodiments, the patient's lower teeth or gums rest in the second groove and the patient's upper teeth or gums rest in the first groove when the body is properly deployed into a patient's mouth. In some embodiments, the first groove extends circumferentially around the body. In some embodiments, either the first or second groove or both comprise a concave indent on the body. In some embodiments, only the patient's front teeth or gums rest in the first groove, the second groove, or the first and second grooves. In some embodiments, the slit intersects the first groove or the second groove or both the first groove and the second groove. In some embodiments, the slit has an anti-pinch feature that protects the patient's lips. In some embodiments the first groove or the second groove or the first groove and the second groove have an anti-pinch feature, such as an anti-pinch feature that protects the patient's lips. In some embodiments, the anti-pinch feature comprises a rounded edge or edges at regions likely to contact a patient's lips when the device is properly deployed. In some embodiments, the body comprises a second set of concave indents to support the patient's lips. Some embodiments lack one or more of the first groove and the second groove.

In some embodiments, the body's shape promotes the anchoring of the bite block in the patient's mouth when the body is properly deployed. In some embodiments this occurs due to a natural tendency of the patient's mouth to rest in a semi-closed position. In some embodiments the patient's teeth or gums then rest on the groove(s) thereby anchoring the construct in the patient's mouth. In some embodiments, the body has an anterior aspect and a posterior aspect. In some embodiments, the grooves are neutral with respect to which teeth they interface with. In some embodiments, the front teeth rest in the grooves. In some embodiments, the central and lateral incisor teeth rest in the grooves. In some embodiments, one or more central and lateral incisor teeth and one or more canine teeth rest in the grooves. In some embodiments, one or more bicuspid teeth rest in the grooves. In some embodiments, one or more molars rest in the grooves. In some embodiments, the body's posterior aspect has a bulbous shape that passively anchors in the mouth by resting proximal to the front teeth or front gums. In some embodiments, the body keeps the patient's mouth open enough to clear the endotracheal tube while minimizing stresses on both the patient's soft tissues and teeth. This embodiment comprises a first groove and a second groove that provide space for the teeth or gums to rest on and further stabilizing the bite block in the anterior-posterior axis. In some embodiments, the ratio of the height of the groove to the height of the bulbous portion is 0.5. In some embodiments, the ratio of the height of the groove to the height of the bulbous portion is no less than 0.01 and no greater than 4.7. In some embodiments, the ratio of the length of the groove to the length of the bulbous portion is 0.33. In some embodiments the ratio of the length of the groove to the length of the bulbous portion is no less than 0.02 and no greater than 1.4. Some embodiments of the body lack a first or a second groove. Accordingly, in some embodiments patient's teeth rest directly on the body or anterior to the body when the body is properly deployed in a patient's mouth. In some embodiments, the hard palate and the tongue contact the body, thereby preventing the upper teeth from contacting the bite block or the lower teeth.

As used herein, a height is in the head-to-toe or superior-to-inferior direction of the patient, when the bite block is properly deployed. As used herein, a width is in the left-to right direction of the patient, when the bite block is properly deployed. As used herein, a length is in the anterior-to-posterior direction of the patient, when the bite block is properly deployed.

The dimensions of the bite block allow the patient's palate to contact the posterior aspect prior to the patient's teeth or gums contacting the grooves in some cases. In some embodiments, contact of the patient's palate to the posterior aspect allows the force of a patient's clenching mouth to be spread over a larger total surface area than if the patient's teeth contacted the grooves prior to the palate contacting the posterior aspect.

In some cases, the bite block has a height that is greater than its width. In some embodiments, the posterior aspect of the bite block is about 1.5 times greater in height than width or the ratio between the height and the width of the posterior aspect of the bite block is about 1.5. In some embodiments, the ratio between the height and the width is about 1.1 to about 4.7. In some embodiments, the height of the posterior aspect of the bite block is great enough to contact the palate of a person receiving the bite block before the person's teeth make contact with each other, while the width of the posterior aspect of the bite block is narrow enough to allow multiple healthcare practitioners to access the mouth of the person.

Often multiple health care practitioners are present during a surgical procedure, including but not limited to one or more surgeons, one or more anesthesiologists, one or more nurses. It is not uncommon for a health care practitioner to require access and/or a view into the patient's mouth.

In some cases, the bite block occupies less than two-thirds of an adult mouth, when properly deployed. In some embodiments, the bite block occupies less than one-third of an adult mouth, when properly deployed. In some embodiments, the width of the bite block is less than one the distance between the bicuspids of an adult mouth. The reduced size relative to bite blocks in the art provides a benefit to a healthcare practitioner since the size of the bite block allows multiple practitioners to access the patient's mouth during a surgical procedure. For example, the size of the bite block allows two practitioners to access the patient's mouth during a surgical procedure. In some embodiments the size of the bite block allows, three practitioners to access the patient's mouth during a surgical procedure. In some embodiments, the size of the bite block allows four practitioners to access the patient's mouth during a surgical procedure. In some embodiments, the size of the bite block allows five or more practitioners to access the patient's mouth during a surgical procedure.

In some cases, the configuration of the grooves allows the bite block to be deployed in multiple positions. In some embodiments, the grooves are neutral with respect to the teeth, allowing any continuous subset of teeth to rest in the groove. In some embodiments, the neutral grooves allow a healthcare practitioner to deploy the bite block into multiple positions. In some embodiments, the neutral grooves allow a healthcare practitioner to deploy the bite block in one configuration and during surgery, when needed, the neutral grooves allow the healthcare practitioner to move the bite block to a second configuration, wherein the first configuration uses a different subset of teeth than the second configuration. In some embodiments, a subset of teeth includes one or more central incisors. In some embodiments, a subset of teeth includes one or more lateral incisors. In some embodiments, a subset of teeth includes one or more cuspids or canines. In some embodiments, a subset of teeth includes one or more bicuspids. In some embodiments, a subset of teeth includes one or more molars. In some embodiments, the bite block protects the teeth, gums, mouth, and endotracheal tube from damage, independent of which teeth or which portion of the gums rest in the grooves of the bite block.

In some cases the body is attached to a tube clamp. In some embodiments the body is reversibly attached to a tube clamp. In some embodiments the body is removed from a tube clamp. In some embodiments the body is detachable from a tube clamp. In some embodiments the body is irreversibly attached to a tube clamp. In some embodiments the body is fabricated in connection with a tube clamp. In some embodiments, the body is not connected to a tube clamp.

### Dimensions of the Bite Block

Bite blocks as disclosed herein are sized to fit into the mouth of a patient. In some cases the bite block has a total volume of less than 3 inches×3 inches×3 inches. In some cases the bite block is sized to provide protection to at least one of the teeth, gums and endotracheal tube, while providing sufficient space in the mouth for the mouth to be accessed by a medical professional, such as an anesthesiologist or other individual benefitting from access to the mouth or oral cavity during a procedure or during intubation. In some cases, the bite block is sized so that it is capable of being moved or repositioned within the mouth of a patient so that different regions of the patient's mouth are capable of being observed or made available for medical procedures during intubation, without a loss in efficacy as a protector against clenching damage.

In some cases, a bite block is sized at about 2.46 inches by 2.5 inches by 2 inches. In some cases the bite block (body) is about 1.5×1.5×2.0 inches. The bulbous portion is about 0.75 inches×1.25 inches×1 inch in some cases). In alternate embodiments, the body is smaller (1 inch×1 inch×1 inch, for example) or larger (3 inches×2 inches×2 inches) depending on the design.

In some embodiments, the bite block is about 2 inches in its height. In some embodiments, the bite block body is about 2.5 inches in its width. In some embodiments, the bite block body is about 2.46 inches in length. In some embodiments, the height of the bite block is no less than 0.75 inches and no greater than 3.5 inches. In some embodiments, the width of the bite block is no less than 0.75 inches and no greater than 3.5 inches. In some embodiments, the length of the bite block is no less than 0.75 inches and no greater than 3.5 inches.

In some embodiments, the bite block body is about 1.5 inches in height. In some embodiments, the bite block body is about 1.5 inches in its width. In some embodiments, the bite block body is about 1.5 inches in its length. In some embodiments, the height of the bite block body is no less than 0.75 inches and no greater than 3.5 inches. In some embodiments, the width of the bite block body is no less than 0.75 inches and no greater than 3.5 inches. In some embodiments, the length of the bite block body is no less than 0.75 inches and no greater than 3.5 inches.

In some embodiments, the posterior part of the bite block body is about 1.5 inches in its height. In some embodiments, the posterior part of the bite block body is about 1.5 inches in it width. In some embodiments, the posterior part of the bite block body is about 1.5 inches in its length. In some embodiments, the height of the posterior part of the bite block body is no less than 0.75 inches and no greater than 3.5 inches. In some embodiments, the width of the posterior part of the bite block body is no less than 0.75 inches and no greater than 3.5 inches. In some embodiments, the length of the posterior part of the bite block body is no less than 0.75 inches and no greater than 3.5 inches.

In some embodiments, the bulbous portion of the posterior part of the bite lock is about 0.75 inches in its length. In some embodiments, the bulbous portion of the posterior part of the bite lock is about 1.25 inches in its width. In some embodiments, the bulbous portion of the posterior part of the bite lock is about 1 inch in its height. In some embodiments, the height of the bulbous portion of the posterior part of the bite block body is no less than 0.75 inches and no greater than 3.5 inches. In some embodiments, the width of the bulbous portion of the posterior part of the bite block body is no less than 0.75 inches and no greater than 3.5 inches. In some embodiments, the length of the bulbous portion of the posterior part of the bite block body is no less than 0.75 inches and no greater than 3.5 inches.

In some embodiments, the anterior aspect of the body is about 1.5 inches in width. In some embodiments, the anterior aspect of the body is about 1.5 inches in height. In some embodiments, the anterior aspect of the body is about 0.75 inches in length. In some embodiments, the width of the anterior aspect is no less than 0.75 inches and no greater than 3.5 inches. In some embodiments, the height of the anterior aspect is no less than 0.75 inches and no greater than 3.5 inches. In some embodiments, the length of the anterior aspect is no less than 0.1 inches and no greater than 2.75 inches.

In some cases, the groove portion of the bite block is about 1.25 inches in width. In some embodiments, the groove portion of the bite block is about 0.5 inches in height. In some embodiments, the groove portion of the bite block is about 0.25 inches in length. In some embodiments, the width of the groove is no less than 0.75 inches and no greater than 3.5 inches. In some embodiments, the height of the groove is no less than 0.05 inches and no greater than 3.5 inches. In some embodiments, the length of the groove is no less than 0.1 inches and no greater than 1.0 inches. In some embodiments, the ratio of the height of the groove to the height of the bulbous portion is 0.5. In some embodiments, the ratio of the height of the groove to the height of the bulbous portion is no less than 0.01 and no greater than 4.7. In some embodiments, the ratio of the length of the groove to the length of the bulbous portion is 0.33. In some embodiments the ratio of the length of the groove to the length of the bulbous portion is no less than 0.02 and no greater than 1.4.

In some cases, the bite block has a height that is greater than its width. In some embodiments, the posterior aspect of the bite block is about 1.5 times greater in height than width or the ratio between the height and the width of the posterior aspect of the bite block is about 1.5. In some embodiments, the ratio between the height and the width is about 1.1 to about 4.7. In some embodiments, the height of the posterior aspect of the bite block is great enough to contact the palate of a person receiving the bite block before the person's teeth make contact with each other, while the width of the posterior aspect of the bite block is narrow enough to allow multiple healthcare practitioners to access the mouth of the person.

As used herein, a height is in the head-to-toe or superior-to-inferior direction of the patient, when the bite block is properly deployed. In some embodiments, a width is in the left-to right direction of the patient, when the bite block is properly deployed. In some embodiments, a length is in the anterior-to-posterior direction of the patient, when the bite block is properly deployed.

In some embodiments, the bite block and its parts are properly sized so that access to the oral cavity with additional medical devices is not blocked. In some embodiments, the bite block and its parts are properly sized so that the positioning of the bite block body is adjustable within the oral cavity. In some embodiments, the position of bite block body is adjustable in the left-to-right direction of the patient after the endotracheal tube is deployed in the bite block. Consistent with embodiments herein, the bite block is multiply deployable in multiple positions within the oral cavity. That is in some embodiments, the bite block is deployable to the center of the mouth. In some embodiments, the bite block is deployed to the left side of the mouth. In some embodiments, the bite block is deployed to the right side of the mouth. In some embodiments, the bite block is initially inserted into the mouth and then re-positioned to another part of the mouth during surgery.

In some embodiments, a bulbous portion lodges in the mouth arrested by teeth or gum lines. In some embodiments, a bulbous portion provides a ball-and-socket type fixation that allows the clamp and body to move within the mouth to a certain level so that it allows the endotracheal tube to be placed at a preferred angle. In some embodiments, the lip portion is part of the bite block body that is anterior to the optional grooves of the bite block body. In some embodiments, the lip portion of the bite block body minimizes potential lip/soft tissue injury because the protective lip of the body directs itself and shield tissue at any angle the endotracheal tube is positioned. In some embodiments, the lip is posterior to the clamp. In some embodiments, the lip is positioned between the lips of the patients and the front teeth and/or gum of the patient.

In some embodiments, the posterior portion of the bite block body is not flat. In some embodiments, the posterior portion of the bite block body has a width that is greater than the cross-sectional diameter of the first channel or an endotracheal tube to be inserted. In some embodiments, the posterior portion of the bite block body has a height that is greater than the diameter of the first channel or an endotracheal tube to be inserted. In some embodiments, the length of the posterior portion of the bite block is similar to the height or width of the posterior part of the bite block. In some embodiments, the posterior part of the bite block is a bulbous shape. In some embodiments, the posterior part of the bite block is a cube. In some embodiments, the posterior part of the bite block is a sphere. In some embodiments, the posterior part of the bite block is a cylinder. In some embodiments, the posterior part of the bite block is a cuboid. In some embodiments, the posterior part of the bite block is a shape of an American football. In some embodiments, the posterior part of the bite block is barrel shaped. In some embodiments, the posterior part of the bite block is a cylindrical segment or a cylinder having a at least one flattened edge. In some embodiments, the posterior part of the bite block is an arbitrary three dimensional shape with a height, a width, and a length greater than the cross-sectional diameter of the first channel.

### Tube Clamp

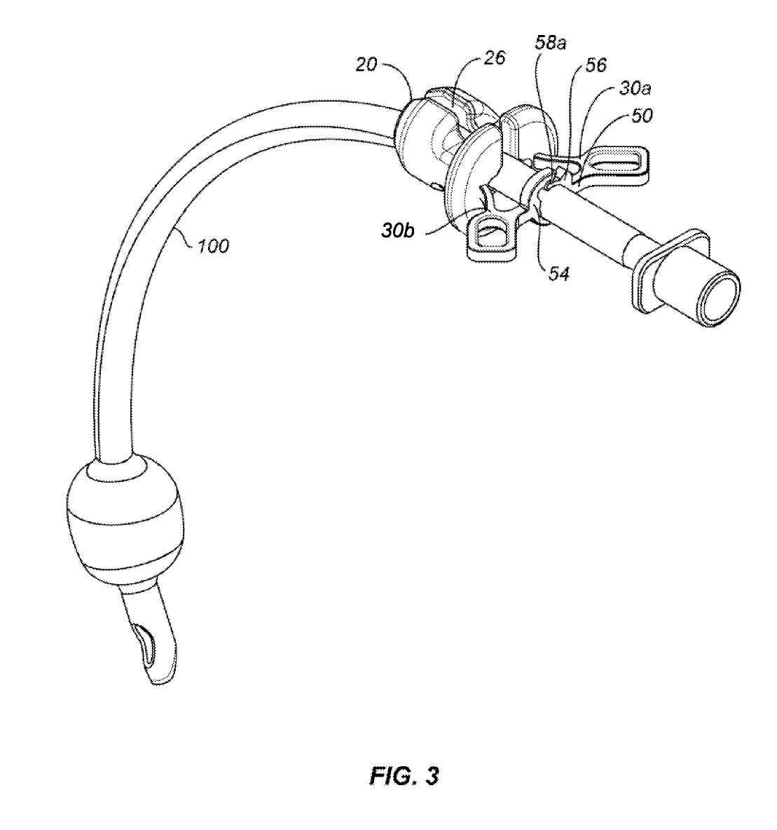

Disclosed herein are bite blocks comprising a body connected to a tube clamp, such as an endotracheal tube clamp. In some embodiments an endotracheal tube clamp clasps an enclosed tube such as an endotracheal tube, holding the attached body in position relative to the endotracheal tube. In some embodiments the size of inner diameter of the tube clamp is tailored to accommodate therein an endotracheal tube such as that depicted in element 100 of FIG. 3, below wherein the endotracheal tube has an inner diameter of 2.5 to 8.5 mm, for example, 2.5, 3.0, 3.5, 4.0, 4.5, 5.0, 5.5, 6.0, 6.5, 7.0, 7.5, 8.0, or 8.5 mm, or any non-integer value within said range of 2.5 to 8.5 mm, at various levels of material interference. In some embodiments the tube clamp interior comprises a plurality of ridges that further clasp a tube such as an endotracheal tube clamped therein.

In some embodiments the tube clamp comprises a latch. Some latch embodiments comprise at least one hook and at least one notch, such that a hook and a notch engage to restrict the inner diameter of the tube clamp. In some embodiments a plurality of notches are provided, thereby allowing for a plurality of tube clamp inner diameters to fit upon engagement of a notch and a latch. In some embodiments, two, three, four, five or more notches are provided, thereby allowing for a plurality of tube clamp inner diameters to fit upon engagement of a notch and a latch. In some embodiments, this feature allows multiple intubation tube diameters to be accommodated by the tube clamp. In some embodiments a tube clamp is secured by a magnet, a plurality of magnets, or a zip clamp such as a reversibly closable zip clamp.

In some embodiments tube clamp components such as a notch and a latch comprise one or more tabs or protrusions or handles through which the notch or latch or both notch and latch are manipulated. In some embodiments such manipulation comprises displacing one or both of the latch and notch laterally with respect to its counterpart such that the latch and notch cease to be engaged. In some embodiments, the endotracheal tube clamp comprises a locking mechanism. In some embodiments, the endotracheal tube clamp has multiple locking mechanisms. In some embodiments, the locking mechanism has a single locking position. In some embodiments, the locking mechanism has multiple locking positions, such as two, three, four, five or more than five locking positions. In some embodiments, the locking mechanism comprises a latch system. In some embodiments, the latch system comprises a hook and a notch that fit together. In some embodiments the latch system comprises a hook with a plurality of notches. In some embodiments the latch system comprises a plurality of hooks. In some embodiments each notch defines a distinct locking position when interfaced with a hook. In some embodiments, the latch comprises a ratcheting system.

In some embodiments, the locking mechanism comprises a handle, or a pair of handles for engaging and disengaging the locking mechanism. In some embodiments, the locking mechanism comprises a pair of handles for engaging and disengaging the locking mechanism. In some embodiments the handles correspond to the neck strap connections.

In some embodiments, the locking mechanism comprises a nut and bolt. In some embodiments, the locking mechanism comprises a magnet, a pair of magnets, or a larger plurality of magnets. In some embodiments, the locking mechanism comprises a zip tie. In some embodiments, the locking mechanism comprises a reversible zip tie. In some embodiments, an interior surface of the endotracheal tube clamp comprises from one to a series of ridges. In some embodiments the series of ridges are capable of griping an enclosed device such as an endotracheal tube when the endotracheal tube clamp is in the locked position. In some embodiments, the endotracheal tube clamp is made of a flexible material such as acetal, nylon, polypropylene, polycarbonate, or acrylic plastic, or other medical grade plastic. In some embodiments the acetal, nylon, polypropylene, polycarbonate, or acrylic plastic, or other medical grade plastic is reinforced with fiberglass reinforcement or other medical grade plastic. In some embodiments the reinforcement is present at 20, 21, 22, 23, 24, 25, 26, 27, 28, 29, 30, 31, 32, 33, 34, 35, 36, 37, 38, 39, or 40%, or any non-integer value within a range of 20% to 40%. In some embodiments the flexible material is nylon, reinforced with 30% fiberglass.

In some embodiments, the tube clamp is attached to at least one neck strap connection. Neck strap connections comprising loops, rectangular openings, slits, or studs are all consistent with embodiments disclosed herein. In some embodiments the neck strap connections comprise loops. In some embodiments the neck strap connections comprise rectangular openings. In some embodiments, the neck strap connections comprise D-shaped openings. In some embodiments the neck strap connections comprise slits. In some embodiments the neck strap connections comprise studs. In some embodiments, a neck strap is deployed to interface with the at least one neck strap connection, such as a pair of neck strap connections on alternate sides of the tube clamp, so as to hold the tube clamp, body and any enclosed tube in place when properly deployed to a patient. In some embodiments the neck strap connections serve as handles that are used to manipulate the tube clamp.

A benefit of a neck strap connection is that it allows the bite block to be secured without it being taped in place. Taping a bite block in place often precludes adjusting the position of the bite block during a procedure, and removing the tape, whether to reposition the block or to remove the block, runs the risk of harming the skin of the patient to which the block is taped.

In some embodiments, the tube clamp, handles and neck strap connections (or neck strap connection/handles, if the elements are convergent) are formed of any solid substance. In some embodiments the above-mentioned components are made of medical grade plastic such as acetal, nylon, polypropylene, polycarbonate, or acrylic plastic, or other medical grade plastic. In some embodiments the medical grade plastic such as acetal, nylon, polypropylene, polycarbonate, or acrylic plastic, or other medical grade plastic is reinforced with fiberglass, such as 20, 21, 22, 23, 24, 25, 26, 27, 28, 29, 30, 31, 32, 33, 34, 35, 36, 37, 38, 39, or 40%, or any non-integer value within a range of 20% to 40%. In some embodiments the flexible material is nylon, reinforced with 30% fiberglass.

In some embodiments the tube clamp is attached to a body. In some embodiments the tube clamp is reversibly attached to a body. In some embodiments the tube clamp is capable of being removed from a body. In some embodiments the tube clamp is detachable from a body. In some embodiments the tube clamp is irreversibly attached to a body. In some embodiments the tube clamp is fabricated in connection with a body.

A benefit of a removable bite block is that a practitioner is able to intubate a patient and then attach the bite block after the tube is properly in place. Also, the bite block is easily removed during surgery, to reposition it or to allow more complete access to a patient's mouth for part of a procedure.

In some embodiments, the bite block comprises finger supports that allow the healthcare provider to open and close the endotracheal tube clamp. In some embodiments, the finger supports are located between the first or second arm and the endotracheal tube clamp. In some embodiments the finger supports are a ring or a pair of rings. In some embodiments the finger supports are rectangular. In some embodiments, pushing the finger supports towards each other locks the endotracheal tube clamp. In some embodiments, the neck strap connection comprises holes or slits located on the finger supports. In some embodiments, the finger supports are rectangular with an opening in the middle. In some embodiments, the neck strap connection is the opening in the finger supports. In some embodiments, the finger supports allow easy locking and unlocking of the endotracheal tube clamp. In some cases finger supports facilitate the locking, unlocking and adjustment of the bite block on the tube.

Some embodiments relate to methods of protecting a patient's mouth or teeth or mouth and teeth from clenching damage during intubation. Some embodiments relate to methods of fixing a tube such as an endotracheal tube in place while protecting a patient's mouth or teeth or mouth and teeth from clenching damage during intubation. Some embodiments comprise positioning a body into a patient's mouth. Some embodiments comprise fixing the body to a tube, such as an endotracheal tube, through a tube clamp. In some embodiments the body is held in place through a neck strap connected to the body, for example through neck strap connections. In some embodiments the body is held in place through a patient's own teeth or gums, which clamp down on the body. In some embodiments a patient's own teeth and gums clamp down on a body through a groove or grooves which assist the patient's own teeth and gums to hold the body in place. In some embodiments the body is positioned within a patient's mouth by a medical professional. In some embodiments the body is positioned to surround a cross section of a tube such as an endotracheal tube deployed into a patient. In some embodiments a tube clasp holds a tube such as an endotracheal tube in position within the mouth of a patient.

In some methods of placement of the bite block, after the patient is intubated, a user such as a healthcare provider opens the endotracheal tube clamp by grasping the arms and applying a bending moment, for example causing the latch disengage as a result of the notch to move laterally along the long axis of the device relative to the hook, to open the endotracheal tube clamp until the endotracheal tube passes through the locking mechanism. Alternately, a user such as a healthcare provider opens the endotracheal tube clamp by grasping the arms and applying a compressing motion to the arms, for example causing the latch to disengage as a result of the notch passing over and along the hook perpendicular to the long axis of the device. Other release methods are contemplated. By opening the endotracheal tube clamp, in some embodiments, the slit also opens to allow the endotracheal tube to reach the first channel in the body. In some embodiments, the body opens because the endotracheal tube clamp is anchored into the lateral aspects of the body, and the first and second arms transfer the bending deflection to the body. Once the endotracheal tube clamp and the body are placed around an endotracheal tube such as the endotracheal tube, in some embodiments the bite block is allowed to translate along the longitudinal axis of the endotracheal tube in the unlocked position. The user such as a healthcare provider then slides the bite block and introduce the body into the patient's mouth in a position that provides the protection of the patient's teeth and soft tissues. Once the desired position is found, in some embodiments the user such as a healthcare provider locks the clamp to secure the position of the bite block relative to the endotracheal tube. In some embodiments, a neck strap is then attached to the neck strap connection to further stabilize the construct by securing the surgical bite block around the patient's neck. In some embodiments, body dimensions such as round body dimensions allow for the surgical bite block to swivel around the mouth in a ball and socket fashion to adjust endotracheal tube position, for example to compensate for an endotracheal tube that is not placed centered with respect to the mouth openings of the patient.

Some embodiments relate to devices for use in protecting a patient's mouth or teeth or mouth and teeth from clenching damage during intubation. Some embodiments relate to devices for use in fixing a tube such as an endotracheal tube in place while protecting a patient's mouth or teeth or mouth and teeth from clenching damage during intubation. Some embodiments comprise positioning a body into a patient's mouth. Some embodiments comprise fixing the body to a tube, such as an endotracheal tube, through a tube clamp. In some embodiments the body is held in place through a neck strap connected to the body, for example through neck strap connections. In some embodiments the body is held in place through a patient's own teeth or gums, which clamp down on the body. In some embodiments a patient's own teeth and gums clamp down on a body through a groove or grooves which assist the patient's own teeth and gums to hold the body in place. In some embodiments the body is positioned within a patient's mouth by a medical professional. In some embodiments the body is positioned to surround a cross section of a tube such as an endotracheal tube deployed into a patient. In some embodiments a tube clasp holds a tube such as an endotracheal tube in position within the mouth of a patient.

In some deployments of a device for use to protect a patient's mouth or teeth or mouth and teeth from clenching damage during intubation, after the patient is intubated, a user such as a healthcare provider opens the endotracheal tube clamp by grasping the arms and applying a bending moment, for example causing the latch disengage as a result of the notch to move laterally along the long axis of the device relative to the hook, to open the endotracheal tube clamp until the endotracheal tube passes through the locking mechanism. Alternately, a user such as a healthcare provider opens the endotracheal tube clamp by grasping the arms and applying a compressing motion to the arms, for example causing the latch to disengage as a result of the notch passing over and along the hook perpendicular to the long axis of the device. Other release methods are contemplated. By opening the endotracheal tube clamp, in some embodiments, the slit also opens to allow the endotracheal tube to reach the first channel in the body. In some embodiments, the body opens because the endotracheal tube clamp is anchored into the lateral aspects of the body, and the first and second arms transfer the bending deflection to the body. Once the endotracheal tube clamp and the body are placed around an endotracheal tube such as the endotracheal tube, in some embodiments, the bite block is allowed to translate along the longitudinal axis of the endotracheal tube in the unlocked position. In some embodiments, the user such as a healthcare provider then slides the bite block and introduce the body into the patient's mouth in a position that provides the protection of the patient's teeth and soft tissues. In some embodiments, the bite block is positioned in multiple locations in the patient's mouth, allowing a healthcare provider to access the mouth and oral cavity. Once the desired position is found, in some embodiments, the user such as a healthcare provider locks the clamp to secure the position of the bite block relative to the endotracheal tube. In some embodiments, a neck strap is then attached to the neck strap connection to further stabilize the construct by securing the surgical bite block around the patient's neck. In some embodiments, body dimensions such as round body dimensions allow for the surgical bite block to swivel around the mouth in a ball and socket fashion to adjust endotracheal tube position, for example to compensate for an endotracheal tube that is not placed centered with respect to the mouth openings of the patient.

### Materials

A number of durable, biocompatible materials are consistent with the embodiments disclosed herein. In some embodiments, the material for forming the body is selected to be durable. Suitable materials include one or more materials are selected to make the body durable, such as durable to sustained clenching pressure as delivered by a clenching human mouth. In some embodiments the materials are able to withstand pressure delivered by clenching top and bottom teeth from an individual intentionally biting down on the material, or subconsciously or unconsciously biting on the material, such as when under sedation accompanying intubation. In some embodiments, one or more materials are selected to facilitate force and pressure distribution. In some embodiments, one or more materials are selected to reduce damage to the mouth, teeth, gums, palate, or combinations thereof. In some embodiments, one or more materials are selected to protect an endotracheal tube intubated in a patient as well as the patient. In some embodiments, the materials for making the body of the bite block includes one or more selected from the list of: a urethane foam, a polyurethane foam, an ethylene foam, a polyethylene foam, a thermoplastic elastomer, a silicone, or a material with characteristics similar thereto. In some embodiments, the body is formed from a plurality of materials, such that an outer layer is biocompatible and such that an inner layer provides durability to pressure exerted on the body by clenching jaws of an individual or patient.

In some embodiments, the material for building the clamp of the bite block comprises one or more materials selected from: nylon, acetyl, polypropylene, polycarbonate, and acrylic, all with or without glass fibers for increased stiffness and/or resilience to the over-molding processes. In some embodiments, the claim is built of a composite of an outer and an inner material, such that the inner material provides rigidity while the outer material protects the inner material from clenching damage. The material is a composite in some cases, such as a nylon composite. The material in some embodiments is a glass composite, comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or greater than 50% glass. In some embodiments the material for building the clamp of the bite block comprises nylon filled with glass with a glass content of 0%-35%.

In some embodiments, the body is made of one or more materials so that the durometer measurement of the body is no less than 10. In some embodiment the durometer measurement of the body is no greater than 80. In some embodiments, the durometer measurement of the body is in the range of 10 to 80. In some embodiments, the body has a durometer measurement of 20. Various embodiments comprise materials having a durometer measurement of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80.

### Benefits to the Mouth of the Material and Dimensions

In some cases the body protects the teeth, gums, or teeth and gums, or teeth, gums, and tongue, cheeks or other soft tissue. The bite block protects the teeth, gums or teeth and gums by distributing clenching force over a larger area of material in the bite block, or a larger area of the gums, teeth, jaw or palate. By partially yielding to the force of clenching jaws, the body provides successively greater surface area in contact with the teeth, gums, palate or other regions attached to the jaws, such that the force is distributed over a successively greater area, and thus pressure, as force per unit area, is distributed or reduced or distributed and reduced. Consequently, in some cases, pressure on the teeth, gums, and mouth does not increase linearly with increase in force exerted by the clenching mouth. In some cases pressure is decreased even as force increases, while in some cases pressure is reduced relative to alternatives that focus clenching force directly on material immediately between the tips of the upper and lower teeth.

In some cases the decrease in pressure at the points of contact between upper and lower teeth protects the teeth from cracking or luxation, protects the gums from bruising, protects the base of the teeth from pressure damage, or protects the gums from bruising. In some cases the decrease in pressure protects the teeth from cracking.

In contrast to devices that direct the teeth into tooth-specific grooves and provide minimal yield in the face of clenching force, the bite block provides an increasing surface area over which a clenching force is distributed in some cases. In many alternative devices, increasing force of clenching results in increasing pressure on the longest teeth, without an increase in surface area, such that the pressure increases linearly with an increase in force, and is often sufficient to damage the teeth, gums, or teeth and gums. The bite block, in contrast, distributes force over an area substantially greater than the point of contact of the longest teeth to the protecting device, and in some cases this area increases as the force increases, such that pressure does not increase linearly with an increase in force to the level necessary to damage the teeth, gums, or teeth and gums, or such that damage to teeth, gums or teeth and gums is substantially reduced. In some cases the force is distributed throughout the teeth rather than at the point of contact, while in some cases the pressure is distributed throughout the teeth and gums, and in some cases the pressure is also distributed across the palate.

In some cases the bite block additionally protects the cheeks of a patient, for example by preventing the jaws from closing on cheek tissue held between upper and lower teeth. Similarly, in some cases the bite block additionally protects the tongue of a patient, for example by preventing the jaws from closing on the tongue held between upper and lower teeth. In some cases the protection is limited to protection of the teeth. In some cases protection is limited to protection of the gums.

Protection is often effected by diffusing the force of clenching jaws over an increased area, thereby decreasing the pressure exerted on the mouth by a given clenching force. In some cases this force is diffused by the use of a body having a durometer value as specified herein. In some cases, this force is diffused by use of the body in which pressure on the teeth, gums and mouth does not increase linearly with increase in force exerted by the clenching mouth.

### Benefits to the Practitioner

In some cases the flexibility of deployment of the bite block allows the bite block to be moved from an area suffering damage in the mouth to an undamaged area. Flexibility of deployment is additionally or alternatively useful in allowing multiple practitioners to access the mouth, allowing multiple tubes to be inserted, and allowing multiple views of the cavity. In some cases, a person undergoing a surgical procedure has multiple practitioners administering care to the person simultaneously during surgery, requiring access to the cavity. In some cases, the flexibility of deployment of the bite block allows two or more practitioners to access the mouth and view the cavity. In some cases, the flexibility of deployment the bite block allows three or more practitioners to access the mouth and view the cavity. In some cases, the flexibility of deployment the bite block allows four or more practitioners to access the mouth and view the cavity. In some cases, the flexibility of deployment of the bite block allows five or more practitioners to access the mouth and view the cavity.

In some cases the bite block is deployable before, during, or after an endotracheal tube or other tube is intubated. That is, the bite block can be attached prior to intubation and simultaneously inserted into the mouth. The bite block can be deployed prior to insertion into the mouth of an endotracheal or other tube.

In a number of exemplary embodiments, the bite block is reversibly deployed after intubation, that is, after a tube is placed into a patient. A benefit of such a feature is that it allows the practitioner to position a tube at his or her convenience within the mouth, emerging straight through the middle of the mouth or on the right or left side, for example, as best suits the medical team. There is also much more flexibility in deploying the tube itself in some cases, such that the medical professional is granted far greater freedom of movement in deploying the tube unhindered by the presence of a securing device. Upon successful deployment, the bite block is used to fix the tube in a desired position in the mouth.

This flexibility in deployment is facilitated by having grooves that are neutral as to the identity or shape of the teeth that they contact. As grooves are not tailored to specific teeth or a specific position in the mouth of a patient, the bite block is deployable in multiple configurations in the mouth of an intubated patient. When the grooves are configured to match a specific set of teeth, deployment is often constrained.

The size of the bite block, and the freedom to deploy it at any position in the mouth and before, during or after intubation also allows a medical practitioner to monitor tube position during a procedure, so that the positioning of the tube can be observed. If necessary, the bite block can be redeployed to adjust the tube position, or the bite block can be removed so that the endotracheal tube or other tube can be repositioned unhindered by a protective device, and then re-fixed in place upon redeployment of the bite block.

Benefits of Securing a Bite Block without Taping

Under a number of standard procedures, a tube such as an endotracheal tube is intubated into a patient and then held in place by fixing the tube using tape attached to the face of the patient. This procedure has at least two detrimental effects. The tape, once in place, is not easily removed if the tube is to be repositioned or if the oral cavity is to be observed accessed. In some embodiments, the tape, when removed, not uncommonly causes damage to the skin to which it was affixed.

The bite block as disclosed herein is reversibly or removably attached to a tube such as an endotracheal tube. The bite block can be attached to a tube after intubation is accomplished, such that the tube is held in place as desired by the medical practitioner using neck straps attached to the device. The bite block is easily removed or repositioned, and need not be removed for the oral cavity to be observed or accessed. Thus in some cases, securing the bite block without tape contributes to the flexibility in deployment that is a benefit of some embodiments of the devices and methods disclosed herein.

In some embodiments, the bite block is positioned in the center of the mouth of the person and then later during the procedure, the bite block is repositioned to the left or right side of the mouth, allowing a medical practitioner to access or view the mouth or oral cavity. In some embodiments, securing the bite block to the person using a neck strap prevents damage to the skin that would be cause if the device was held in place using tape.

### Slit Orientation

In some embodiments, the bite block is partially or fully divided by a slit, such as a slit facilitating deployment and removal of the bite block from a tube. In some cases the slit bisects the top groove, bisects the bottom groove, or bisects the top groove and bottom groove. In some embodiments, the slit bisects the lip. The lip of the bite block, in some embodiments, is a portion that is posterior to the clamp and anterior to the top and bottom grooves. In some embodiments, the lip of the bite block is placed in between the lips and the front teeth and/or gums of a patient, when the bite block is properly deployed. In some embodiments, the slit bisects the bulbous portion of the bite block body. In some embodiments, the slit bisects the bite block body. In some embodiments, the slit bisects the posterior part of the bite block. ‘Bisects’ can be interpreted to mean, in some cases, dividing into equal or approximately equal parts. In some cases bisects can be interpreted to divide into two parts, independent of the relative proportions of the two parts.

In some embodiments, the slit is vertical relative to the ‘horizontal’ plane of a patient's upper and lower jaws. In some embodiments, the slit is positioned at an angle relative to the central vertical plane of the bite block of anywhere from 0 degrees to 90 degrees. In some embodiments, the slit is positioned at an angle relative to a plane defined by the left-to right axis and the anterior-to posterior axis of the patient, wherein the angle is anywhere from 0 degrees to 90 degrees.

In some embodiments, the slit runs through the bite block body. In some embodiments, the slit is a continuous slit starting at the lip of the bite block body. In some embodiments, the slit runs through the top or bottom groove of the bite block body. In some embodiments, the slit runs through the posterior part of the body.

In embodiments having a slit, the slit facilitates attachment and removal of a bite block from an endotracheal tube. Thus the practitioner has the flexibility to attach a bite block after intubation, remove and reattach it during a procedure or remove it prior to removal of the tube.

In some embodiments, the slit connects to the first channel throughout the bite block body. In some embodiments, the lit connects to the first channel all the way throughout the anterior-to-posterior axis of the patient starting at the lip the bite block body and ending in the most posterior section of the bulbous portion of the bite block.

In some embodiments, the posterior aspect is anchored proximally posterior to the front teeth or gum of the patient when deployed. In some embodiments, at least part of the posterior aspect is in contact with the palate of the mouth. In some embodiments, at least part of the posterior aspect is in contact with the tongue. In some embodiments, the posterior aspect facilitates pressure or force absorption by providing additional contacting surface to the bite block. In some embodiments, the posterior aspect facilitates pressure or force absorption by its non-permanent deformation. In some embodiments, the deformation of the bite block body induced by biting force or biting pressure is reversible.

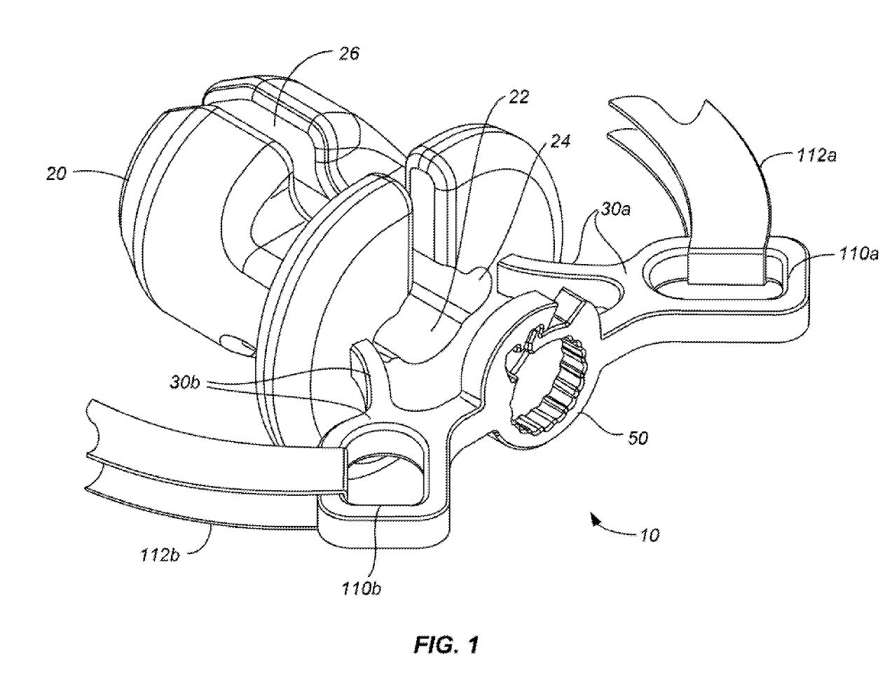

FIG. 1 shows an exemplary embodiment of a bite block 10. In some embodiments, a bite block 10 comprises a body 20 and an endotracheal tube clamp 50. In some embodiments, the body 20 comprises any shape that will fit within a patient's mouth. In some embodiments, the body 20 is spherical. In some embodiments, the body 20 is cylindrical. In some embodiments the body 20 is ovoid. In some embodiments, alternative body shapes consistent with the disclosure herein share the element of capability of being inserted into the mouth cavity of a patient. In some embodiments any shape consistent with this element is contemplated. In some embodiments, the body 20 is made of a soft material. In some embodiments, the body 20 is made of a soft material such as Silicone or a medical grade foam. In some embodiments, the body 20 comprises a first channel 22 running through the body 20. In some embodiments, the first channel 22 is sized and shaped to allow a tube (such as an endotracheal tube as depicted in element 100 of FIG. 3, below) to pass through the first channel 22. In some embodiments the diameter of the first channel 22 ranges from 2 to 15 mm, for example 2, 3, 4, 5, 6, 7, 8, 9, 10, 11, 12, 13, 14, or 15 mm, or any non-integer value within said range of 2 to 15 mm. In some embodiments the size of the first channel 22 is tailored to accommodate therein an endotracheal tube such as that depicted in element 100 of FIG. 3, below wherein the endotracheal tube has an inner diameter of 2.5 to 8.5 mm, for example, 2.5, 3.0, 3.5, 4.0, 4.5, 5.0, 5.5, 6.0, 6.5, 7.0, 7.5, 8.0, or 8.5 mm, or any non-integer value within said range of 2.5 to 8.5 mm, at various levels of material interference. In some embodiments, the first channel 22 is sized, shaped or sized and shaped to allow at least one other device such as at least one temperature probe to pass through the first channel 22. In some embodiments, the first channel 22 is circular in cross section. In some embodiments, the first channel 22 is elliptical in cross section. In some embodiments the first channel is triangular, rectangular, pentagonal, hexagonal, heptagonal, octagonal, or demonstrates alternative regular or irregular geometry in cross section. In some embodiments, only an endotracheal tube such as that seen as element 100 of FIG. 3 can pass through the first channel 22. In some embodiments, other instruments can be placed through the first channel 22 alongside the endotracheal tube 100. In some embodiments, the body 20 comprises at least a second channel 24 or a plurality of channels. In some embodiments the second channel 24 is connected to the first channel 22. In some embodiments, the second channel 24 intersects the first channel 22. In some embodiments the second channel 24 allows at least one other device such as at least one temperature probe to pass through the body 20. In some embodiments, the bite block 10 comprises a slit 26 on the body 20. In some embodiments the slit 26 is coplanar with the first channel 22. In some embodiments the slit 26 is on the superior side of the body 20. In some embodiments, the slit 26 is on the inferior side of the body 20. In some embodiments, the slit 26 extends into the first channel 22. In some embodiments the slit allows an endotracheal tube such as the endotracheal tube 100 of FIG. 3, below, or other devices to slide through the slit 26 and into the first channel 22. In some embodiments, the slit 26, intersects the second channel 24. In some embodiments, the slit 26 extends completely through the first channel 22 and divides the body into two pieces. In some embodiments the two pieces of the body 20 are held together by the endotracheal tube clamp 50. A view of an embodiment of an endotracheal tube clamp 50 separate from a body is provided in FIG. 7, further discussed below. In some embodiments, the body 20 connects to the endotracheal tube clamp 50 at a first arm 30a or a first arm 30a and a second arm 30b. In some embodiments the body 20 is molded around the first arm 30a or the first and second arms 30a and 30b. In some embodiments, the first arm 30a or the first and second arms 30a and 30b are attached to the body 20 with one or more screws. In some embodiments, the first arm 30a or the first and second arms 30a and 30b are attached to the body with an adhesive. In some embodiments, the first arm 30a or the first and second arms 30a and 30b are made of a flexible material. In some embodiments the first arm 30a or the first and second arms 30a and 30b are made of a flexible material such as acetal nylon, polypropylene, polycarbonate, or acrylic plastic, or other medical grade plastic. In some embodiments the acetal nylon, polypropylene, polycarbonate, or acrylic plastic, or other medical grade plastic is reinforced with fiberglass reinforcement or other medical grade plastic. In some embodiments the reinforcement is present at 20, 21, 22, 23, 24, 25, 26, 27, 28, 29, 30, 31, 32, 33, 34, 35, 36, 37, 38, 39, or 40%, or any non-integer value within a range of 20% to 40%. In some embodiments the flexible material is nylon, reinforced with 30% fiberglass. In some embodiments, the first arm 30a or the first and second arms 30a and 30b are shaped and positioned such that they allow concurrent opening of the endotracheal tube clamp 50 and the body 20, for example to ease the placement of the endotracheal tube. In some embodiments the first arm 30a or the first and second arms 30a and 30b are curved. In some embodiments, the first arm 30a or the first and second arms 30a and 30b are S shaped. In some embodiments, the device comprises a neck strap having ends seen at 112a, 112b and a neck strap connection 110a, 110b. In some embodiments, the neck strap connection 110a, 110b is attached to the first arm 30a or the first and second arms 30a and 30b. In some embodiments, the neck strap connection 110a is built into the first arm 30a. A number of neck strap configurations are consistent with the disclosure herein. In some embodiments, the neck strap connections are each a ring. In some embodiments the neck strap connections are each rectangular. In some embodiments, the neck strap connections are each a clip. In some embodiments, the neck strap connections are each a post. In some embodiments, the neck strap having ends seen at 112a, 112b fits around the patient's neck to further secure the device to the patient.

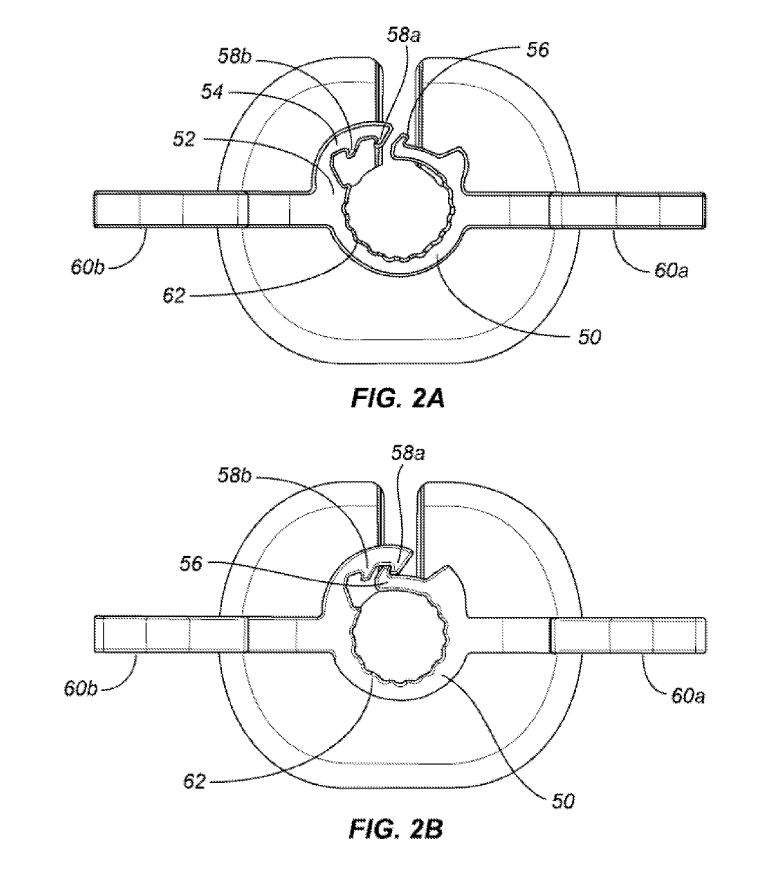

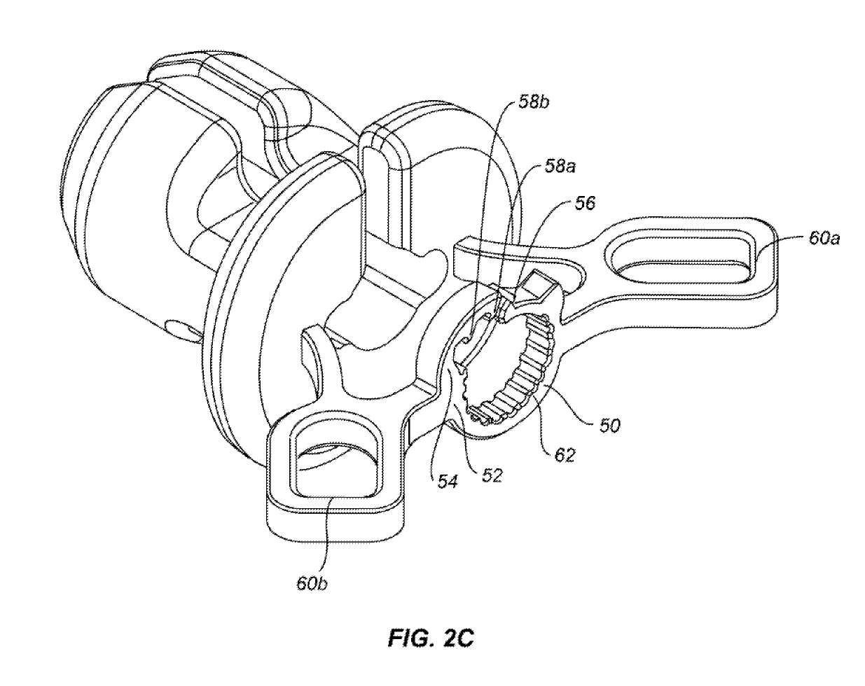

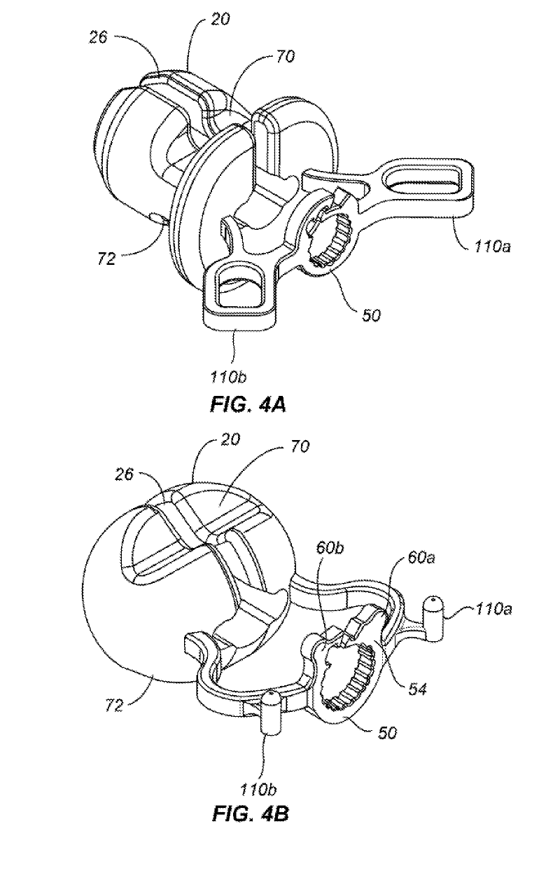

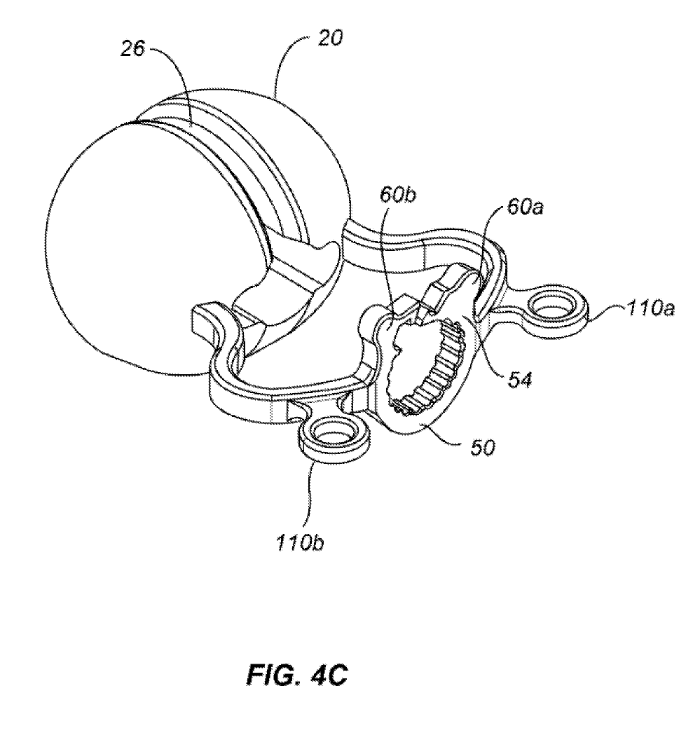

FIGS. 2A, 2B and 2C show alternate views and alternate configurations of an embodiment of the endotracheal tube clamp 50 joined to a body such as element 20 of FIG. 1, above. In some embodiments, the endotracheal tube clamp comprises a locking mechanism 52. In some embodiments, the endotracheal tube clamp 50 has multiple locking mechanisms 52. In some embodiments, the locking mechanism 52 has a single locking position. In some embodiments, the locking mechanism 52 has multiple locking positions. In some embodiments, the locking mechanism 52 comprises a latch system 54. In some embodiments, the latch system 54 comprises a hook 56 and a notch 58a that fit together. In some embodiments the latch system 54 comprises a hook 56 with a plurality of notches 58a, 58b. In some embodiments the latch system 54 comprises a plurality of hooks 56. In some embodiments each notch 58a-b defines a distinct locking position when interfaced with a hook 56. FIG. 2A shows a latch system 54 wherein a hook 56 is disengaged from notches 58a and 58b. FIG. 2B shows a latch system 54 wherein a hook 56 is engaged with a notch 58a in a first locked position. A second locked position for said latch system, not shown, is assumed wherein a hook 56 is engaged with a notch 58b. In some embodiments, the locking mechanism 52 comprises a handle 60a, or a pair of handles 60a and 60b for engaging and disengaging the locking mechanism 52. In some embodiments, the locking mechanism 52 comprises a pair of handles 60a, 60b for engaging and disengaging the locking mechanism 52. In some embodiments the handles 60a, 60b correspond to the neck strap connections 110a, 110b of FIG. 1, above. Alternate handle embodiments are presented in FIGS. 4A-C and 5A-B, and alternate handle embodiments not depicted therein are also contemplated. In some embodiments, the locking mechanism 52 comprises a nut and bolt. In some embodiments, the locking mechanism 52 comprises a magnet, a pair of magnets, or a larger plurality of magnets. In some embodiments, the locking mechanism 52 comprises a zip tie. In some embodiments, the locking mechanism 52 comprises a reversible zip tie. In some embodiments, an interior surface of the endotracheal tube clamp 50 comprises from one to a series of ridges 62. In some embodiments the series of ridges 62 can grip an enclosed device such as the endotracheal tube 100 of FIG. 3 when the endotracheal tube clamp 50 is in the locked position. In some embodiments, the endotracheal tube clamp 50 is made of a flexible material such as acetal, nylon, polypropylene, polycarbonate, or acrylic plastic, or other medical grade plastic. In some embodiments the acetal, nylon, polypropylene, polycarbonate, or acrylic plastic, or other medical grade plastic is reinforced with fiberglass reinforcement or other medical grade plastic. In some embodiments the reinforcement is present at 20, 21, 22, 23, 24, 25, 26, 27, 28, 29, 30, 31, 32, 33, 34, 35, 36, 37, 38, 39, or 40%, or any non-integer value within a range of 20% to 40%. In some embodiments the flexible material is nylon, reinforced with 30% fiberglass. FIG. 2A shows an embodiment of an endotracheal tube clamp in an unlocked position. In some embodiments, the healthcare provider locks the clamp by pushing one handle 60 towards the other, which causes the latch 54 to engage and the endotracheal tube clamp 50 to become locked. FIG. 2B shows an embodiment of an endotracheal tube clamp 50 in an unlocked position. In some embodiments, the endotracheal tube clamp 50 applies a compression force to the endotracheal tube 100 of FIG. 3 when the endotracheal tube clamp 50 is in the locked position. In some embodiments, a user such as a healthcare provider unlocks the clamp by applying a force to the handles 60a, 60b pushing one handle in the posterior direction and the other in the anterior direction causing the engaged latch 54 such as that of FIG. 2A to disengage, assuming the configuration of the disengaged latch 54 of FIG. 2B, and the endotracheal tube clamp 50 to release. In some embodiments, a user such as a healthcare provider twists the handles 60 in opposite directions to release the endotracheal tube clamp 50. In some embodiments, the endotracheal tube clamp 50 is unlocked by twisting either clockwise or counterclockwise.

FIG. 3 shows the placement of an embodiment of the bite block 10 around an endotracheal tube 100. In one method of placement of the bite block 10, after the patient is intubated, a user such as a healthcare provider opens the endotracheal tube clamp 50 by grasping the arms 30a, 30b and applying a bending moment, for example causing the latch 54 disengage as a result of the notch 58a or 58b to move laterally along the long axis of the device 10 relative to the hook 56, to open the endotracheal tube clamp 50 until the endotracheal tube 100 can pass through the locking mechanism 52. Alternately, a user such as a healthcare provider opens the endotracheal tube clamp 50 by grasping the arms 30a, 30b and applying a compressing motion to the arms 30a, 30b, for example causing the latch 54 to disengage as a result of the notch 58a or 58b passing over and along the hook 56 perpendicular to the long axis of the device 10. Other release methods are contemplated. By opening the endotracheal tube clamp, in some embodiments, the slit 26 also opens to allow the endotracheal tube 100 to reach the first channel in the body 20. In some embodiments, the body 20 opens because the endotracheal tube clamp 50 is anchored into the lateral aspects of the body 20, and the first and second arms 30a, 30b transfer the bending deflection to the body 20. Once the endotracheal tube clamp 50 and the body 20 are placed around an endotracheal tube such as the endotracheal tube 100, in some embodiments the bite block is allowed to translate along the longitudinal axis of the endotracheal tube 100 in the unlocked position. In some embodiments, the user such as a healthcare provider then slides the bite block 10 and introduce the body 20 into the patient's mouth in a position that provides the protection of the patient's teeth and soft tissues. Once the desired position is found, in some embodiments, the user such as a healthcare provider locks the clamp to secure the position of the bite block 10 relative to the endotracheal tube 100. In some embodiments, a neck strap is then attached, for example as seen in 112a, 112b of FIG. 1, to the neck strap connection 110a, 110b of FIG. 1 to further stabilize the construct by securing the surgical bite block around the patient's neck. In some embodiments, body dimensions such as round body dimensions allow for the surgical bite block to swivel around the mouth in a ball and socket fashion to adjust endotracheal tube position, for example to compensate for an endotracheal tube that is not placed centered with respect to the mouth openings of the patient.

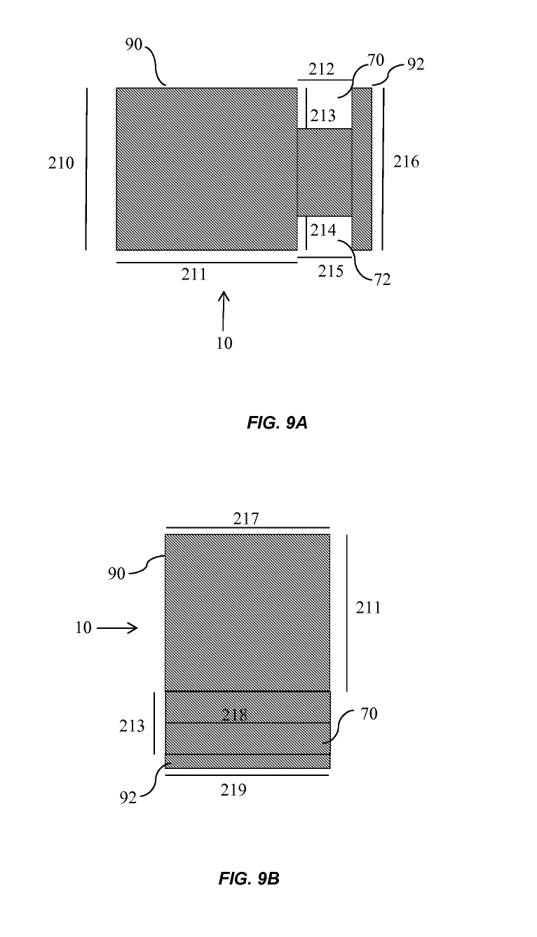

FIGS. 4A-C show embodiments of the bite block 10. In some embodiments the bite block 10 when properly deployed protects a patent's teeth, jaws or mouth from clenching damage. In some embodiments, such as those seen in FIGS. 4A-4B, the bite block 10 comprises a body with a first groove 70. In some embodiments, the first groove 70 has a length represented by 212 in FIG. 9A. In some embodiments, the first groove 70 has a height represented by 213 in FIG. 9A. In some embodiments, the first groove 70 has a width represented by 218 in FIG. 9B. In some embodiments, the patient's teeth or gums rest within the first groove 70, further securing the body 20 in the patient's mouth when the body 20 is properly deployed into a patient's mouth. In some embodiments, the body has a second groove 72. In some embodiments, the second groove 72 has a length represented by 214 in FIG. 9A. In some embodiments, the second groove 72 has a height represented by 215 in FIG. 9A. In some embodiments, the patient's lower teeth or gums rest in the second groove 72 and the patient's upper teeth and gums rest in the first groove 70 when the body 20 is properly deployed into a patient's mouth. In some embodiments, the first groove 70 extends circumferentially around the body 20. In some embodiments, either the first or second groove 70, 72 or both comprise a concave indent on the body 20. In some embodiments, the slit 26 intersects the first groove 70 or the second groove 72 or both the first groove and the second groove. In some embodiments, the slit 26 has an anti-pinch feature that protects the patient's lips. In some embodiments, the anti-pinch feature comprises a rounded edge or edges at regions likely to contact a patient's lips when the device is properly deployed. In some embodiments, the body 20 comprises a second set of concave indents to support the patient's lips. As seen in FIG. 4C, some embodiments lack one or more of the first groove 70 and the second groove 72

FIGS. 4A-C further shows embodiments of neck strap connections. A number of embodiments of neck strap connections are contemplated, as are embodiments lacking neck straps connections. In some embodiments, neck strap connections is configured as rectangular or rounded rectangular structures, as in FIG. 4A elements 110a, 110b. In some embodiments, neck strap connections are configured as a post or posts, as in FIG. 4B elements 110a, 110b, which can be inserted into a hole in a neck strap. In some embodiments, neck strap connections are configured as circular or ring-like structures, as in FIG. 4C elements 110a, 110b, through which a neck strap is threaded.

FIGS. 4A-C also vary in body structure. In some embodiments, any body structure is combined with any neck strap connection, or no neck strap connection, in embodiments herein. In some embodiments the neck strap connection comprises a slit in each of the connecting arms 30a, 30b. In some embodiment the neck strap connection comprises a post 110a, 110b that can be inserted into a hole in the neck strap.

FIGS. 4B and 4C also depict alternate placements of handles 60a, 60b as nobs affixed to the latching mechanism to facilitate engagement and disengagement of the latch.

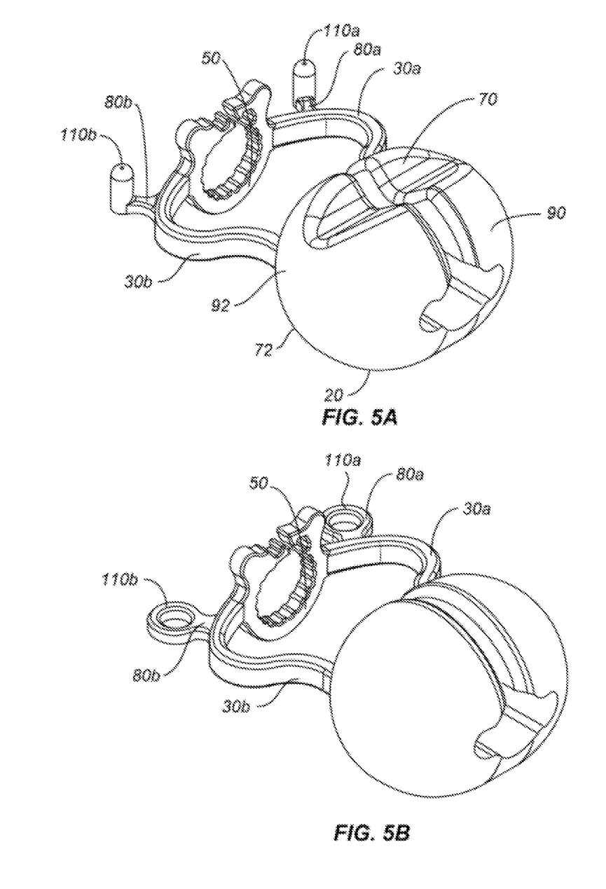

FIGS. 5A-B show embodiments of the body 20. In some embodiments, the body's 20 shape promotes the anchoring of the bite block 10 in the patient's mouth when the body is properly deployed. In some embodiments, this occurs due to a natural tendency of the patient's mouth to rest in a semi-closed position. In some embodiments, the patient's teeth or gums then rest on the groove(s) thereby anchoring the construct in the patient's mouth. As seen in FIG. 5AB, in some embodiments, the body 20 has an anterior aspect 92 and a posterior aspect 90. In some embodiments, the anterior aspect 92 has a height represented by 216 in FIG. 9A. In some embodiments, the anterior aspect 92 has a width represented by 219 in FIG. 9B. In some embodiments, the posterior aspect 90 has a height represented by 210 in FIG. 9A. In some embodiments, the posterior aspect 90 has a length represented by 211 in FIG. 9A. In some embodiments, the posterior aspect 90 has a width represented by 217 in FIG. 9B. In some embodiments, the body's 20 posterior aspect 90 has a bulbous shape that passively anchors in the mouth by resting proximal to the front teeth or front gums. In some embodiments, the body 20 keeps the patient's mouth open enough to clear the endotracheal tube 100 while minimizing stresses on both the patient's soft tissues and teeth. This embodiment comprises a first groove 70 and a second groove 72 that provide space for the teeth or gums to rest on and further stabilizing the bite block 10 in the anterior-posterior axis, as depicted in FIG. 5A. As seen, for example, in FIG. 5B, some embodiments of the body 20 lack a first or a second groove. Accordingly, in some embodiments patient's teeth rest directly on the body or anterior to the body when the body is properly deployed in a patient's mouth.

In some embodiments, the bite block 10 comprises finger supports, such as 80a, 80b seen in FIG. 5A, that allow the healthcare provider to open and close the endotracheal tube clamp. In some embodiments, the finger supports 80a, 80b are located between the first or second arm 30a, 30b and the endotracheal tube clamp 50. In some embodiments the finger supports 80a, 80b are a ring. In some embodiments the finger supports 80a, 80b are rectangular. In some embodiments, pushing the finger supports 80a, 80b towards each other locks the endotracheal tube clamp 50. In some embodiments, the neck strap connection, such as 110a, 110b comprises holes or slits located on the finger supports 80a, 80b. In some embodiments, the finger supports 80a, 80b are rectangular with an opening in the middle. In some embodiments, the neck strap connection such as 110a, 110b is the opening in the finger supports 80a, 80b. In some embodiments, the finger supports allow easy locking and unlocking of the endotracheal tube clamp 50.

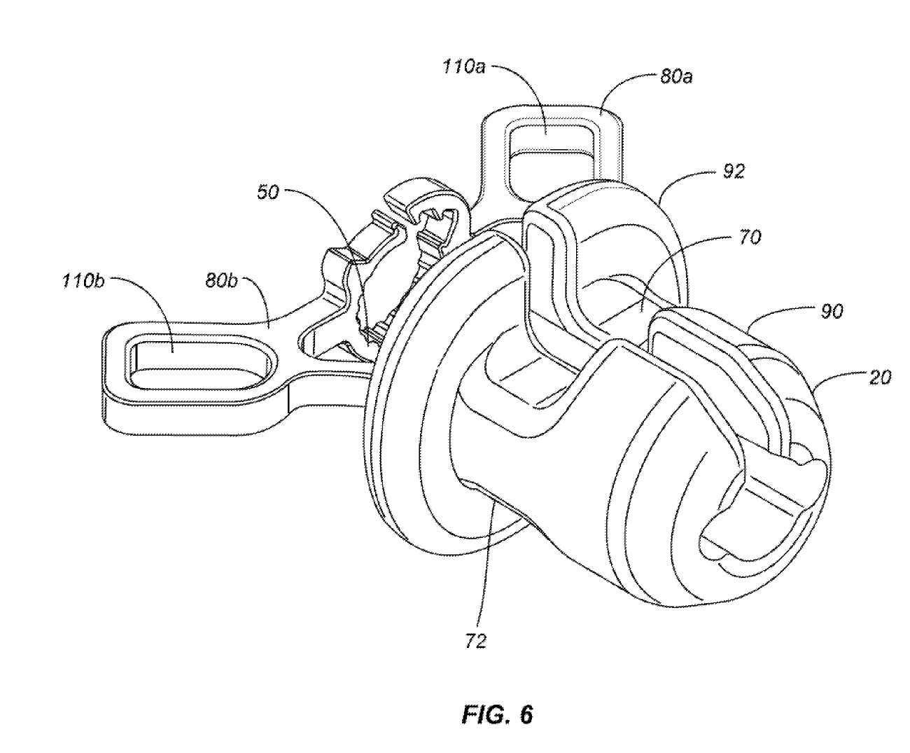

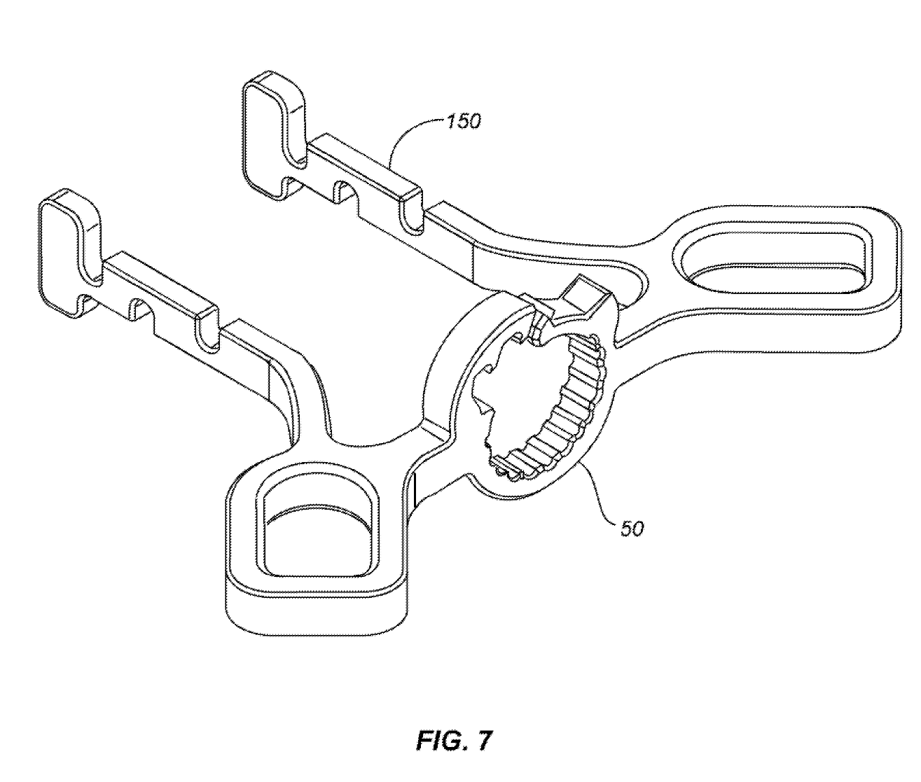

FIG. 6 shows an embodiment of the body 20 with a bulbous shaped posterior aspect 90. In some embodiments, the posterior aspect 90 has a height represented by 210 in FIG. 9A. In some embodiments, the posterior aspect 90 has a length represented by 211 in FIG. 9A. In some embodiments, the posterior aspect 90 has a width represented by 217 in FIG. 9B. In some embodiments, the bulbous shaped posterior aspect 90 is combined with a cylindrical shaped anterior aspect 92. In some embodiments the anterior aspect is rectangular shaped or ovoid. In some embodiments the anterior aspect is bisected by a slit such as element 26 of FIG. 1.

FIG. 7 shows an isolated view of the endotracheal tube clamp 50 with features as indicated in the discussion of FIG. 1, above, showing struts 150 through which the clamp is affixed to a body such as the body in FIG. 1.

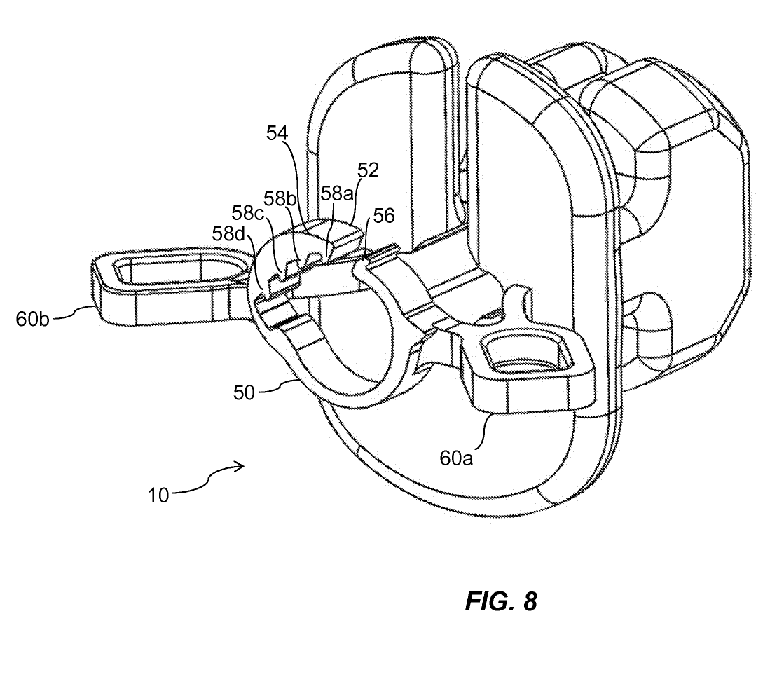

FIG. 8 shows an alternative view of a bite block 10, wherein the endotracheal tube clamp 50, which is in an unlocked position has multiple locking positions 52. In this view, a latch system 54 comprises a hook 56 which is disengaged from notches 58a, 58b, 58c and 58d. Four distinct locking positions for said latch system is assumed wherein a hook 56 is engaged with any of notches 58a, 58b, 58c or 58d. Also shown is a pair of handles 60a and 60b for engaging and disengaging the locking mechanism 52.

FIG. 9A and FIG. 9B show dimensions of various elements of a bite block without the endotracheal tube clamp. In FIG. 9A, the posterior aspect 90 has a height 210 and a length 211. Also in FIG. 9A, the first groove 70 has a length 212 and a height 213. In this view, the second groove 72 has a length 215 and a height 214. Also in FIG. 9A, the anterior aspect has a height 216. In some embodiments, the posterior aspect 90 has a length 211 at least as long as the length 212 of the first groove 70 or the length 215 of the second groove 72. In some embodiments, the posterior aspect 90 has a height 210 at least two times the height 213 of the first groove 70 or the height 214 of the second groove 72.

In FIG. 9B, the posterior aspect 90 has a width 214 and a length 211. Also in FIG. 9B, the first groove 70 has a width 218 and a length 213. Also in FIG. 9B, the anterior aspect has a width 219.

As defined herein, “about” a scalar quantity refers to that quantity plus or minus 10%.

“Person” or “patient” as used herein, means an individual undergoing a medical procedure, optionally under general anesthesia and optionally intubated, wherein the person or patient is at risk of clenching damage. In some embodiments, the person is an adult. “Mouth” as used herein, means the teeth and gums, palate, and optionally includes soft tissues associated therewith such as cheeks, lips and tongue. ‘Mouth’ refers in some cases to the teeth, gums, and palate of a patient. In some cases the bite block protects the mouth of a patient from clenching damage.

“Adult mouth” as used herein refers to an average adult mouth with average distance between the lips and the throat, an average distance between the bicuspid teeth, and an average distance between the tongue and the palate.

“In the mouth” as used herein refers to a position partially or totally interior to or between the upper and lower teeth, gums, or teeth and gums, optionally in contact with the palate.

“Oral cavity” as used herein refers to the space within the mouth, up to an including the throat in some cases.

“Clenching force” as used herein refers to the force of a human lower jaw against an upper jaw when the two are drawn together, as in when biting or when the jaw muscles are otherwise brought to bear. Clenching in some cases is conscious or intentional, or in other cases is unconscious or unintentional, as in the case of a patient that unconsciously clenches his or her jaws when under sedation. Clenching one's teeth similarly refers to the application of clenching force.

Clenching force results in a pressure distributed over the area of contact of the teeth or gums of the upper and lower jaws, either with one another or with an intervening object. Increasing the area over which the clenching force is applied will decrease the pressure per unit area, as pressure refers to force per unit area.

Area of clenching contact refers to the area contacted by the teeth, gums, or teeth and gums, of a clenching upper and lower jaw. In some cases the area of clenching contact is beneficially be expanded to include the palate or additional teeth and gum area not otherwise contacted during clenching. When intubated, in some embodiments, a patient's area of clenching is limited to the teeth, gum or teeth and gum region immediately adjacent to the tube such as an endotracheal tube, concentrating the clenching force on a reduced area.

As used herein unless otherwise noted, horizontal refers to a plane that is parallel to the upper and lower jaw of a patient. Vertical refers to a plane that is perpendicular to a plane that is parallel to the upper and lower jaw of a patient. In some cases, ‘vertical’ is parallel to the height of the patient, while horizontal is parallel to a line running through the width of a patient.

### EXAMPLES

### Example 1

A bite block as disclosed herein is used in a patient in need of endotracheal intubation. The endotracheal tube is inserted into the patient, and the bite block is properly deployed in the mouth of the patient following a successful intubation procedure to protect the endotracheal tube from being occluded and to protect the patient from clenching damages to the mouth, the teeth, the gums, the palate, or any other tissue within the mouth. The procedure is repeated in 10 different patient.

The bite block contacts a large area of the mouth: the lip of the bite block contacts the teeth and gums and inner side of the lips, the grooves contacts the front teeth and gums, the posterior part of the bite lock contact the palate and the tongue. All the parts of the bite block body increase the contacting surface area over which clenching force is distributed, thereby reducing pressure on any given part of the mouth from clenching. In addition to the increased surface, the bite block body as disclosed herein deforms and provides a cushion when the patient bites. No noticeable damage is observed in patients using the bite block disclosed herein. A traditional mouth guard is also used for 10 other patients in need of intubation. The patients with traditional mouth guard are observed to suffer a substantially greater pressure at the point of contact between the jaws and the protector. Accordingly, patients using traditional mouth guard are observed to suffer clenching damage at a greater frequency during intubation procedures.

### Example 2

A bite block as disclosed herein is used in a patient in need of endotracheal intubation. The endotracheal tube is inserted into the patient, and the bite block is properly deployed in the mouth of the patient following a successful intubation procedure to protect the endotracheal tube from being occluded and to protect the patient from clenching damages to the mouth, the teeth, the gums, the palate, or any other tissue within the mouth. The procedure is repeated in 10 different patients. A traditional mouth guard is also used for 10 other patients in need of intubation.

Practitioners using the bite block as described herein are able to reposition the bite block to access the interior of the oral cavity and to observe the position of the endotracheal tube. Displaced endotracheal tubes are easily adjusted, and additional tubes are easily deployed. The bite block is easily removed and redeployed as necessary to adjust the endotracheal tube.

Practitioners using a traditional mouth guard are unable to observe to access the interior of the mouth of a patient in which a mouth guard is deployed. When the interior of the oral cavity is to be accessed, the guard must be removed. The endotracheal tube is only positioned in a single configuration, and options for accessing the oral cavity are curtailed.

It is observed that procedures which are undertaken using the bite block are finished more quickly and with fewer complications due to the ease of accessing the interior of the oral cavity and the ease of viewing and repositioning the endotracheal tube.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A bite block comprising:
a body comprising a posterior aspect, a first groove, a second groove, and an anterior portion;
a first channel running through the body from an external side of the body to a mouth side of the body through the posterior aspect, the first channel being sized to accommodate an endotracheal tube therein; and
a slit extending from the first channel to an outer surface of the body through the posterior aspect, such that the bite block can be deployed onto an endotracheal tube after the endotracheal tube is intubated into a person;
wherein the posterior aspect has a length along the first channel at least as long as the width of the first groove along the first channel, and wherein the posterior aspect has a height at least two times the height of the first groove.

2. The bite block of claim 1, comprising an endotracheal tube clamp coupled to the body, sized to accommodate an endotracheal tube therein.

3. The bite block of claim 1, wherein the bite block occupies less than two-thirds of an adult mouth opening.

4. The bite block of claim 1, wherein the bite block is operably deployable in a first position and a second position within the mouth of the person.

5. The bite block of claim 1, wherein the bite block, when operably deployed into the mouth of the person, does not block access or line of sight to the oral cavity of the person by a health care professional.

6. The bite block of claim 1, wherein the first groove and the second groove are neutral with respect to teeth that rest in the groove.

7. The bite block of claim 1, wherein the posterior aspect distributes clenching force beyond an initial point of contact between the person's upper and lower teeth, gums, or teeth and gums.

8. The bite block of claim 1, comprising a second channel.

9. A method for protecting teeth and gums from clenching damage in a person requiring endotracheal tube administration, comprising the steps of
surrounding a segment of an endotracheal tube using a bite block having a posterior aspect such that the endotracheal tube passes through the posterior aspect; and
deploying the bite block to the interior of the person's mouth,
wherein the posterior aspect distributes clenching force over an area of teeth, gums, teeth and gums, or palate of the person that is greater than an area of clenching contact between the person's upper and lower jaws and a bite block lacking a posterior aspect during intubation.

10. The method of claim 9, wherein the bite block contacts the palate of the person before the bite block contacts the teeth of the person in an unclenching mouth.

11. The method of claim 9, wherein the posterior aspect is compressed before the person's teeth contact the bite block.

12. The method of claim 9, wherein the clenching force is distributed over a first area at a first force of clenching and the clenching force is distributed over a second area at a second force of clenching, wherein the second force of clenching is greater than the first force of clenching, and wherein increase in pressure per unit area is not linear with increasing force.

13. A device for the mouth of an intubated person, the device comprising a first groove configured to fit any adjacent subset of teeth of the person, a posterior body, and a channel through the posterior body configured to accommodate an endotracheal tube.

14. The device of claim 13, wherein the posterior body has a length measured parallel to the channel at least as long as the width of the first groove measured parallel to the channel and has a height at least two times the height of the first groove.

15. The device of claim 13 comprising an endotracheal tube clamp coupled to the anterior body, said endotracheal tube clamp sized to accommodate an endotracheal tube therein.

16. The device of claim 13, comprising a slit extending from the first channel to an outer surface of the device through the posterior body, such that the device is deployable onto an endotracheal tube after the endotracheal tube is intubated into the person.

17. The device of claim 13, wherein the device occupies less than two-thirds of an adult mouth opening when deployed into an adult mouth such that teeth of the adult mouth fit in the first groove.

18. The device of claim 13, wherein the device, when operably deployed into the mouth of the person, does not block access or line of sight to the oral cavity of the person by a health care professional.

19. The device of claim 13, wherein the posterior body and the first groove distribute clenching force over an area of teeth, gums, teeth and gums, or palate of the person that is greater than an area of clenching contact between the person's upper and lower jaws.

20. The device of claim 13, wherein the posterior body comprises a shape having each dimension greater than the cross-sectional diameter of the first channel.
